(19) United States
(12) Patent Application Publication
(10) Pub. No.: US 2026/0022362 A1
(43) Pub. Date: Jan. 22, 2026

(54) RECOMBINANT IMMUNOGLOBULIN G-SPECIFIC ENDOGLYCOSIDASES, PHARMACEUTICAL COMPOSITIONS, AND USES THEREOF

(71) Applicant: Emory University, Atlanta, GA (US)

(72) Inventors: Eric SUNDBERG, Decatur, GA (US); Diego SASTRE, Sao Paulo (BR)

(21) Appl. No.: 19/275,445

(22) Filed: Jul. 21, 2025

Related U.S. Application Data
(60) Provisional application No. 63/673,353, filed on Jul. 19, 2024.

(51) Int. Cl. C12N 9/24

(52) U.S. Cl. CPC C12N 9/2402; C12Y 302/01096; C07K 2319/30

(57) ABSTRACT

This disclosure relates to recombinant immunoglobulin endoglycosidases. In certain embodiments, the recombinant immunoglobulin endoglycosidases comprise insertions and/or mutations disclosed herein. In certain embodiments, this disclosure relates to pharmaceutical compositions comprising recombinant immunoglobulin endoglycosidases disclosed herein and/or nucleic acids or vector encoding the same. In certain embodiments, this disclosure relates to pharmaceutical compositions. In certain embodiments, this disclosure relates to recombinant immunoglobulin G-specific endoglycosidase CU43 comprising a dipeptide FK insertion at position 240 wherein the dipeptide FK insertion is in reference to positions in the CP40 amino acid sequence.

### CROSS REFERENCE TO RELATED APPLICATION[S]

This application claims priority to, and the benefit of, U.S. Provisional Patent Application Ser. No. 63/673,353 filed on Jul. 19, 2024, the entire contents of which are incorporated herein by reference as if set forth in its entirety.

### STATEMENT REGARDING FEDERALLY SPONSORED RESEARCH OR DEVELOPMENT

This invention was made with government support under AI149297 awarded by the National Institutes of Health. The government has certain rights in the invention.

### SEQUENCE LISTING

The instant application contains a Sequence Listing which has been submitted electronically in .XML format and is hereby incorporated by reference in its entirety. Said.XML copy, created on Jul. 21, 2025, is named “080503-1040 Sequence Listing.xml” and is 151,552 bytes in size.

### BACKGROUND

Humans produce antibodies containing immunoglobulin G (IgG) proteins which are abundantly present in the blood. As part of the immune system, IgG antibodies play a central role in protecting the body from pathogens by recognizing the presence of biological material that is not a “self-molecule.” When IgG antibodies specifically bind a pathogen, a series of biological events occur that cause the body to attack the pathogen. However, environmental factors, genetic defects, and aging sometimes inadvertently directs these same events to occur abnormally. Thus, the presence of undesirable IgG antibodies are often the cause of autoimmune diseases and inflammatory disorders. Recombinant therapeutic antibody therapies exist for certain autoimmune diseases and inflammatory disorders; however, treatments are not always universally effective and sometimes have side effects. Thus, there is a need to identify improvements.

Human IgG proteins contain variable regions and constant regions (Fc). The Fc region contains an asparagine amino acid at position 297, (Asn297 or N297), which is commonly substituted to contain different chains of sugar units, also referred to as a glycan. The state of glycosylation at Asn297 is reported to be involved in regulating immunological events.

Flevaris et al. report immunoglobulin G N-glycan biomarkers for autoimmune diseases. Int J Mol Sci, 2022, 23, 5180

Shadnezhad et al. report CP40 from Corynebacterium pseudotuberculosis is an endo-β-N-acetylglucosaminidase. BMC Microbiology, 2016, 16:261.

Yamin et al. report human FcγRIIIa activation on splenic macrophages drives dengue pathogenesis in mice. Nat Microbiol, 2023, 8(8): 1468-1479.

Gupta et al. report mechanisms of glycoform specificity and in vivo protection by an anti-afucosylated IgG nanobody. Nat Commun, 2023, 14:2853.

Bournazos et al. report antibody fucosylation predicts disease severity in secondary dengue infection. Science, 2021, 372(6546): 1102-1105.

Sastre D E, Bournazos S, Du J, Boder E J, Edgar J E, Azzam T, Sultana N, Huliciak M, Flowers M, Yoza L, Xu T, Chernova T A, Ravetch J V, Sundberg E J. Potent efficacy of an IgG-specific endoglycosidase against IgG-mediated pathologies. Cell. 2024 Nov. 27;187(24):6994-7007.e12. doi: 10.1016/j.cell.2024.09.038. Epub 2024 Oct. 21. PMID: 39437779; PMCID: PMC11606778.

Sastre, D. E., et al. The mechanistic basis for interprotomer deglycosylation of antibodies by corynebacterial IgG-specific c endoglycosidases. Nat Commun 16, 6147 (2025). https://doi.org/10.1038/s41467-025-60986-w.

References cited herein are not an admission of prior art.

### SUMMARY

This disclosure relates to immunoglobulin G-specific endoglycosidases and uses in managing diseases or conditions associated with immune dysregulation. In certain embodiments, this disclosure relates to methods of cleaving or altering glycans attached to immunoglobulins comprising contacting a glycosylated immunoglobulin and an immunoglobulin G-specific endoglycosidase disclosed herein providing a glycan cleaved immunoglobulin.

In certain embodiments, this disclosure relates to recombinant immunoglobulin G-specific endoglycosidase CU43 comprising a dipeptide FK insertion at position 240 wherein the dipeptide FK insertion is in reference to positions in the CP40 amino acid sequence.

ATLSKEPLKASPGRADTVGVQTTCNAKPIFFGYYRTWRDKAIQLKDDDPWKDKL QVKLTDIPEHVNMVSLFHVEDNQKSDQQFWETFHREYQPELKKRGTRVVRTVGAQLLL NKIKDKNLYGKHVEDDYKYREIARDVYNEYVVKHNLDGLDVDMELRQVEKQLNLKW QLRKIMGAFSELMGPKAPANEGKKPDHEGYKYLIYDTFDNAQTSQVGLVADLVDYVLA QTYKKDTKESVTQVWNGFRDKINSCQFMAGYAHPEENDTNRFLTAVGEVNKSGAMQV AEWKPEGGEKGGTFAYALDRDGRTYDGDDFTTLKPTDFAFTKRAIELTTGESSTDLGKP TGSR (SEQ ID NO: 57) wherein the N-terminal amino acid alanine (A) is position 34.

In certain embodiments, this disclosure relates to recombinant immunoglobulin G-specific endoglycosidases comprising an E mutation at position 263 in reference to positions in the CP40 amino acid sequence (SEQ ID NO: 57), wherein the N-terminal amino acid alanine (A) is position 34.

In certain embodiments, a mutation in position D263 in which the amino acid D (Aspartic Acid) is changed to E (Glutamic Acid) in reference to positions in the CP40 amino acid sequence (SEQ ID NO: 57), wherein the N-terminal amino acid alanine (A) is position 34.

In certain embodiments, this disclosure relates to recombinant immunoglobulin G-specific endoglycosidases comprising sequences disclosed herein. In certain embodiments, this disclosure relates to nucleic acids and vectors encoding immunoglobulin endoglycosidases disclosed herein.

In certain embodiments, this disclosure relates to pharmaceutical compositions comprising recombinant immunoglobulin endoglycosidases disclosed herein and/or nucleic acids or vector encoding the same.

In certain embodiments, the recombinant immunoglobulin G-specific endoglycosidase comprises an FK peptide insertion at position 240 providing a peptide motif of the following amino acid sequence FFKD (SEQ ID NO: 50). In certain embodiments, the recombinant immunoglobulin G-specific endoglycosidase further comprises an E amino acid at amino acid position 263 providing a peptide motif of the following amino acid sequence TYEK (SEQ ID NO: 51). In certain embodiments, the recombinant immunoglobulin G-specific endoglycosidase comprises peptide motifs of the following amino acid sequences FFKD (SEQ ID NO: 50) and TYEK (SEQ ID NO: 51).

In certain embodiments, the recombinant immunoglobulin G-specific endoglycosidase comprises peptide motifs of the following amino acid sequences from an N-terminal to C-terminal order sequentially, YYRTWRDK (SEQ ID NO: 1), TDIP, (SEQ ID NO: 2), LDGLDX¹DME (SEQ ID NO: 3), FFKD (SEQ ID NO: 50), TYEK (SEQ ID NO: 51), SCQF (SEQ ID NO: 4), NRFL (SEQ ID NO: 5), and DRDG (SEQ ID NO: 6), wherein X¹ is I or V or any amino acid.

In certain embodiments, the recombinant immunoglobulin G-specific endoglycosidase does not contain an amino acid sequence KESV (SEQ ID NO: 48, contained in CP40) between TYEK (SEQ ID NO: 51) and SCQF (SEQ ID NO: 4).

In certain embodiments, the recombinant immunoglobulin G-specific endoglycosidase comprises peptide motifs of the following amino acid sequences from an N-terminal to C-terminal order sequentially, AXXXXXPLXXXXGXXXXXG (SEQ ID NO: 7), PIXXXYYRTWRDKXIXXXXXD (SEQ ID NO: 8), TDIPXXXXXXSLXHVXD, (SEQ ID NO: 9), QXSDXXFWXTFXXXYXPXLXXRGTXVXXTXXXXXXL (SEQ ID NO: 10), YXXXAXXXXXXYVXXHXLDGLDXDME, (SEQ ID NO: 11), LRXXMXAXXXLXGPXXXXN (SEQ ID NO: 12), LXYXTFFKDNAQXXXXXXV (SEQ ID NO: 56), AXXVXYVXXQTYEK (SEQ ID NO: 18), WNXXRXXXXSCQFXXXYAXPEEXDX (SEQ ID NO: 15), NRFLXAXGXGXVXXXXAXXXAXWXP (SEQ ID NO: 16), and GXKGGXXXYAXDRDGXTYDXXDXXTLXXTXFXXXKR (SEQ ID NO: 17), wherein X is individually and independently at each position any amino acid or variant thereof.

In certain embodiments, the recombinant immunoglobulin G-specific endoglycosidase comprises the amino acid sequence (CU43_ins240FK_D263E):

(SEQ ID NO: 54)
APAALSNAPLAASPGQADKVGAQATCAAKPIFFGYYRTWRDKAIE
LNDGDKWKDKLHTKLTDIPEQVDMVSLFHVPDNQKSDQRFWETFD
KEYHPTLKERGTKVVRTIGAKLLLNKIKEKGLYGQSREDDSKYRE
IAHEVYEEYVAKHNLDGLDVDMELREVEKYTNLRWQLRKIMGAFS
ELMGPKAPGNAGKKPGDDGYKYLIYDTFFKDNAQLAQVALVADVV
DYVLAQTYEKGTEESITRVWNGFRDKINSCQFLAGYAHPEENDTN
RFLTAIGDVDTSGAMNVAAWKPEGGEKGGTFAYALDRDGRTYDGD
DLTTLKPTDFAFTKRAIELTKGISLTDLGTNGSRTPGANSSRTET
SSFFNDPYYQKLMKEAGIADNTN.

In certain embodiments, the recombinant immunoglobulin G-specific endoglycosidase comprises the amino acid sequence of:
MRNVPQSVSRFATVGITSAIFAASLSTVASAAPAALSNAPLAASPGQADKVGA QATCAAKPIFFGYYRTWRDKAIELNDGDKWKDKLHTKLTDIPEQVDMVSLFHVPDNQK SDQRFWETFDKEYHPTLKERGTKVVRTIGAKLLLNKIKEKGLYGQSREDDSKYREIAHE VYEEYVAKHNLDGLDVDMELREVEKYTNLRWQLRKIMGAFSELMGPKAPGNAGKKP GDDGYKYLIYDTFFKDNAQLAQVALVADVVDYVLAQTYEKGTEESITRVWNGFRDKI NSCQFLAGYAHPEENDTNRFLTAIGDVDTSGAMNVAAWKPEGGEKGGTFAYALDRDG RTYDGDDLTTLKPTDFAFTKRAIELTKGISLTDLGTNGSRTPGANSSRTETSSFFNDPYYQ KLMKEAGIADNTN (SEQ ID NO: 47) or without the signal sequence MRNVPQSVSRFATVGITSAIFAASLSTVASA (SEQ ID NO: 49) providing:
APAALSNAPLAASPGQADKVGAQATCAAKPIFFGYYRTWRDKAIELNDGDKWK DKLHTKLTDIPEQVDMVSLFHVPDNQKSDORFWETFDKEYHPTLKERGTKVVRTIGAK LLLNKIKEKGLYGQSREDDSKYREIAHEVYEEYVAKHNLDGLDVDMELREVEKYTNLR WQLRKIMGAFSELMGPKAPGNAGKKPGDDGYKYLIYDTFFKDNAQLAQVALVADVV DYVLAQTYEKGTEESITRVWNGFRDKINSCQFLAGYAHPEENDTNRFLTAIGDVDTSGA

MNVAAWKPEGGEKGGTFAYALDRDGRTYDGDDLTTLKPTDFAFTKRAIELTKGISLTD LGTNGSRTPGANSSRTETSSFFNDPYYQKLMKEAGIADNTN (SEQ ID NO: 54).

In certain embodiments, the recombinant immunoglobulin G-specific endoglycosidase comprises an FF peptide insertion at position 240 providing a peptide motif of the following amino acid sequence FFFD (SEQ ID NO: 20).

In certain embodiments, the recombinant immunoglobulin G-specific endoglycosidase comprises peptide motifs of the following amino acid sequences from an N-terminal to C-terminal order sequentially, YYRTWRDK (SEQ ID NO: 1), TDIP, (SEQ ID NO: 2), LDGLDX¹DME (SEQ ID NO: 3), FFFD (SEQ ID NO: 20), SCQF (SEQ ID NO: 4), NRFL (SEQ ID NO: 5), and DRDG (SEQ ID NO: 6), wherein X¹ is I or V or any amino acid.

In certain embodiments, the recombinant immunoglobulin G-specific endoglycosidase does not contain an amino acid sequence KESV (SEQ ID NO: 48, contained in CP40) between FFFD (SEQ ID NO: 20) and SCQF (SEQ ID NO: 4).

In certain embodiments, the recombinant immunoglobulin G-specific endoglycosidase comprises peptide motifs of the following amino acid sequences from an N-terminal to C-terminal order sequentially, AXXXXXPLXXXXGXXXXXG (SEQ ID NO: 7), PIXXXYYRTWRDKXIXXXXXD (SEQ ID NO: 8), TDIPXXXXXXSLXHVXD, (SEQ ID NO: 9), QXSDXXFWXTFXXXYXPXLXXRGTXVXXTXXXXXXL (SEQ ID NO: YXXXAXXXXXXYVXXHXLDGLDXDME, (SEQ ID NO: 11), LRXXMXAXXXLXGPXXXXN (SEQ ID NO: 12), LXYXTFFFDNAQXXXXXXV (SEQ ID NO: 21), WNXXRXXXXSCQFXXXYAXPEEXDX (SEQ ID NO: 15), 10), NRFLXAXGXGXVXXXXAXXXAXWXP (SEQ ID NO: 16), and GXKGGXXXYAXDRDGXTYDXXDXXTLXXTXFXXXKR (SEQ ID NO: 17), wherein X is individually and independently at each position any amino acid or variant thereof.

In certain embodiments, this disclosure relates to fusion proteins comprising any of the immunoglobulin G-specific endoglycosidases as provided herein. In certain embodiments, the fusion of immunoglobulin G-specific endoglycosidases as provided herein and a Fc domain.

In certain embodiments, this disclosure relates to a nucleic acid or vector encoding a recombinant immunoglobulin G-specific endoglycosidase as provided herein in operable combination with a heterologous promoter. In certain embodiments, this disclosure relates to vectors comprising a nucleic acid or encoding a recombinant immunoglobulin G-specific endoglycosidase as provided herein. In certain embodiments, this disclosure relates to attenuated bacterial strains (non-naturally occurring) such as attenuated Escherichia coli (E. coli) strains comprising a nuclei acid encoding a recombinant immunoglobulin G-specific endoglycosidase as provided herein, i.e., heterologous expression. In certain embodiments, the nucleic acid is mRNA, RNA, or DNA.

### BRIEF DESCRIPTION OF THE DRAWINGS

FIG. 1A shows a sequence comparison of CU43 (SEQ ID NO: 19, Q1) and CP40 (SEQ ID NO: 57, S1).

FIG. 1B shows a sequence comparison of CU43_ins240FK_D263E (SEQ ID NO: 54,

Query, Q) and CU43 (SEQ ID NO: 19, Subject, S).

FIG. 2A illustrates the glycol structures of an antibody and Fc with fucosylated (with triangle fucose) and afucosylated (without triangle) modifications at Asn297.

FIG. 2B shows data on the relative changes in deglycosylation over time when exposed immunoglobulin G (IgG) fucosylated or afucosylated to CU43 which shows similar preference for fucosylated and afucosylated Fc-IgG1.

FIG. 2C shows data on the relative changes in deglycosylation over time when exposed to immunoglobulin G (IgG) fucosylated or afucosylated to CU43 with an FF insertion at position 240 (CU43_ins240FF).

FIG. 2D shows data on the relative changes in deglycosylation over time when exposed immunoglobulin G (IgG) fucosylated or afucosylated to CU43 with an FK insertion after amino acid F at position 240 and a D to E mutation at position 263 (CU43_ins240FK_D263E).

FIG. 2E shows data indicating that after consumption of the IgG afucosylated diglycosylated species, approximately 70% of the completely glycosylated Fc-fucosylated intact species remained.

### DETAILED DISCUSSION

It is to be understood that the terminology used herein is for the purpose of describing particular embodiments only, and is not intended to be limiting, since the scope of the present disclosure will be limited only by the appended claims or as amended during prosecution.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

An “embodiment” of this disclosure refers to an example, but not necessarily limited to such example. As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, immunology and the like, which are within the skill of the art. Such techniques are explained fully in the literature. It must be noted that, as used in the specification and the appended claims, the singular forms “a,” “an,” and “the” include plural referents unless the context clearly dictates otherwise. In this specification and in the claims that follow reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods.

A “subject” refers to any animal, preferably a human patient, livestock, or domestic pet.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the terms “prevent” and “preventing” include the prevention of the recurrence, spread or onset. It is not intended that the present disclosure be limited to complete prevention. In some embodiments, the onset is delayed, or the severity of the disease is reduced. A “nucleic acid” refers to a DNA- or RNA-molecule and is used synonymous with polynucleotide. Wherever herein reference is made to a nucleic acid or nucleic acid sequence encoding a particular protein and/or peptide, said nucleic acid or nucleic acid sequence, respectively, preferably also comprises regulatory sequences allowing in a suitable host, e.g., a human being, its expression, i.e., transcription and/or translation of the nucleic acid sequence encoding the particular protein or peptide.

“Amino acid sequence” is defined as a sequence composed of any one of the 20 naturally appearing amino acids, amino acids which have been chemically modified, or composed of synthetic amino acids. The terms “protein” and “peptide” refer to compounds comprising amino acids joined via peptide bonds and are used interchangeably. As used herein, where “amino acid sequence” is recited herein to refer to an amino acid sequence of a protein molecule. An “amino acid sequence” can be deduced from the nucleic acid sequence encoding the protein. Furthermore, unless the context demands otherwise, the term “peptide” and “polypeptide” and “protein” are used interchangeably to refer to amino acids in which the amino acid residues are linked by covalent peptide bonds or alternatively (where post-translational processing has removed an internal segment) by covalent disulfide bonds, etc. The ammo acid chains can be of any length and comprise at least three amino acids, they can include domains of proteins or full-length proteins. Unless otherwise stated the terms peptide, polypeptide, and protein also encompass various modified forms thereof, including but not limited to glycosylated forms, phosphorylated forms, etc.

The term “comprising” in reference to a peptide having an amino acid sequence refers a peptide that may contain additional N-terminal (amine end) or C-terminal (carboxylic acid end) amino acids, i.e., the term is intended to include the amino acid sequence within a larger peptide. The term “consisting of” in reference to a peptide having an amino acid sequence refers a peptide having the exact number of amino acids in the sequence and not more or having not more than a range of amino acids expressly specified in the claim. In certain embodiments, the disclosure contemplates that the “N-terminus of a peptide consists of an amino acid sequence,” which refers to the N-terminus of the peptide having the exact number of amino acids in the sequence and not more or having not more than a range of amino acids specified in the claim; however, the C-terminus may be connected to additional amino acids, e.g., as part of a larger peptide. Similarly, the disclosure contemplates that the “C-terminus of a peptide consists of an amino acid sequence,” which refers to the C-terminus of the peptide having the exact number of amino acids in the sequence and not more or having not more than a range of amino acids specified in the claim; however, the N-terminus may be connected to additional amino acids, e.g., as part of a larger peptide. In certain embodiments, this disclosure relates to proteins disclosed herein consisting of sequences disclosed herein having less than an additional 10, 50, 100, or 200 amino acids on the N-terminus. In certain embodiments, this disclosure relates to proteins disclosed herein consisting of sequences disclosed herein having less than an additional 10, 50, 100, or 200 amino acids on the C-terminus.

The term “recombinant” when made in reference to a nucleic acid molecule refers to a nucleic acid molecule that is comprised of segments of nucleic acids joined together by means of molecular biological techniques. The term “recombinant” when made in reference to a protein or a polypeptide refers to a protein molecule that is expressed using a recombinant nucleic acid molecule.

A “heterologous” nucleic acid sequence or peptide sequence refers to a nucleic acid sequence or a peptide sequence that does not naturally occur, e.g., because the whole sequence contains a segment from other plants, bacteria, viruses, other organisms, or joinder of two sequences that occur the same organism but are joined together in a manner that does not naturally occur in the same organism or any natural state.

The terms “vector” or “expression vector” refer to a recombinant nucleic acid containing a desired coding sequence and appropriate nucleic acid sequences necessary for the expression of the operably linked coding sequence in a particular host organism or expression system, e.g., cellular or cell-free. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

Protein “expression systems” refer to in vivo (e.g. host cell) and in vitro (cell free) systems. Systems for recombinant protein expression typically utilize somatic cells transfecting with a DNA or mRNA expression vector that contains the template. The cells are cultured under conditions such that they translate the desired protein. Expressed proteins are extracted for subsequent purification. In vivo protein expression systems using prokaryotic and eukaryotic cells are well known. Proteins may be recovered using denaturants and protein-refolding procedures. In vitro (cell-free) protein expression systems typically use translation-compatible extracts of whole cells or compositions that contain components sufficient for transcription, translation, and optionally post-translational modifications such as RNA polymerase, regulatory protein factors, transcription factors, ribosomes, tRNA cofactors, amino acids, and nucleotides. In the presence of an expression vectors, these extracts and components can synthesize proteins of interest. Cell-free systems typically do not contain proteases and enable labeling of the protein with modified amino acids. Some cell free systems incorporated encoded components for translation into the expression vector. See, e.g., Shimizu et al., Cell-free translation reconstituted with purified components, 2001, Nat. Biotechnol, 19, 751-755 and Asahara & Chong, Nucleic Acids Research, 2010, 38(13): e141, both hereby incorporated by reference in their entirety.

A “selectable marker” is a nucleic acid introduced into a recombinant vector that encodes a polypeptide that confers a trait suitable for artificial selection or identification (report gene), e.g., beta-lactamase confers antibiotic resistance, which allows an organism expressing beta-lactamase to survive in the presence antibiotic in a growth medium. Another example is thymidine kinase, which makes the host sensitive to ganciclovir selection. It may be a screenable marker that allows one to distinguish between wanted and unwanted cells based on the presence or absence of an expected color. For example, the lac-z-gene produces a beta-galactosidase enzyme which confers a blue color in the presence of X-gal (5-bromo-4-chloro-3-indolyl-β-D-galactoside). If recombinant insertion inactivates the lac-z-gene, then the resulting colonies are colorless. There may be one or more selectable markers, e.g., an enzyme that can complement to the inability of an expression organism to synthesize a particular compound required for its growth (auxotrophic) and one able to convert a compound to another that is toxic for growth. URA3, an orotidine-5′ phosphate decarboxylase, is necessary for uracil biosynthesis and can complement ura3 mutants that are auxotrophic for uracil. URA3 also converts 5-fluoroorotic acid into the toxic compound 5-fluorouracil. Additional contemplated selectable markers include any genes that impart antibacterial resistance or express a fluorescent protein. Examples include, but are not limited to, the following genes: ampr, camr, tetr, blasticidinr, neor, hygr, abxr, neomycin phosphotransferase type II gene (nptII), p-glucuronidase (gus), green fluorescent protein (gfp), egfp, yfp, mCherry, p-galactosidase (lacZ), lacZa, lacZAM15, chloramphenicol acetyltransferase (cat), alkaline phosphatase (phoA), bacterial luciferase (luxAB), bialaphos resistance gene (bar), phosphomannose isomerase (pmi), xylose isomerase (xylA), arabitol dehydrogenase (atID), UDP-glucose: galactose-1-phosphate uridyltransferasel (galT), feedback-insensitive α subunit of anthranilate synthase (OASA1D), 2-deoxyglucose (2-DOGR), benzyladenine-N-3-glucuronide, E. coli threonine deaminase, glutamate 1-semialdehyde aminotransferase (GSA-AT), D-amino acidoxidase (DAAO), salt-tolerance gene (rstB), ferredoxin-like protein (pflp), trehalose-6-P synthase gene (AtTPS1), lysine racemase (lyr), dihydrodipicolinate synthase (dapA), tryptophan synthase beta 1 (AtTSB1), dehalogenase (dhlA), mannose-6-phosphate reductase gene (M6PR), hygromycin phosphotransferase (HPT), and D-serine ammonialyase (dsdA).

A “label” refers to a detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. A label includes the incorporation of a radiolabeled amino acid or the covalent attachment of biotinyl moieties to a polypeptide that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as ¹⁸F, ³⁵S or ¹³¹I) fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms of various lengths to reduce potential steric hindrance.

In certain embodiments, the disclosure relates to recombinant polypeptides comprising sequences disclosed herein or variants or fusions thereof wherein the amino terminal end or the carbon terminal end of the amino acid sequence are optionally attached to a heterologous amino acid sequence, label, or reporter molecule.

In certain embodiments, the disclosure relates to the recombinant vectors comprising a nucleic acid encoding a polypeptide disclosed herein or chimeric/fusion protein thereof.

In certain embodiments, the recombinant vector optionally comprises a mammalian, human, insect, viral, bacterial, bacterial plasmid, yeast associated origin of replication or gene such as a gene or retroviral gene or lentiviral LTR, TAR, RRE, PE, SLIP, CRS, and INS nucleotide segment or gene selected from tat, rev, nef, vif, vpr, vpu, and vpx or structural genes selected from gag, pol, and env.

In certain embodiments, the recombinant vector optionally comprises a gene vector element (nucleic acid) such as a selectable marker region, lac operon, a CMV promoter, a hybrid chicken B-actin/CMV enhancer (CAG) promoter, tac promoter, T7 RNA polymerase promoter, SP6 RNA polymerase promoter, SV40 promoter, internal ribosome entry site (IRES) sequence, cis-acting woodchuck post regulatory element (WPRE), scaffold-attachment region (SAR), inverted terminal repeats (ITR), FLAG tag coding region, c-myc tag coding region, metal affinity tag coding region, streptavidin binding peptide tag coding region, polyHis tag coding region, HA tag coding region, MBP tag coding region, GST tag coding region, polyadenylation coding region, SV40 polyadenylation signal, SV40 origin of replication, Col El origin of replication, f1 origin, pBR322 origin, or pUC origin, TEV protease recognition site, loxP site, Cre recombinase coding region, or a multiple cloning site such as having 5, 6, or 7 or more restriction sites within a continuous segment of less than 50 or 60 nucleotides or having 3 or 4 or more restriction sites with a continuous segment of less than 20 or 30 nucleotides.

The term “fusion” when used in reference to a polypeptide refers to the expression product of two or more coding sequences obtained from different sources such that they do not exist together in a natural environment, that have been cloned together and that, after translation, act as a single polypeptide sequence. The coding sequences include those obtained from the same or from different species of organisms.

In certain embodiments, this disclosure relates to recombinant immunoglobulin G-specific endoglycosidases comprising a dipeptide FK insertion at position 240 wherein the dipeptide FK insertion is in reference to positions in amino acid sequence (SEQ ID NO: 19). wherein the N-terminal amino acid alanine (A) is position 32. In certain embodiments, this disclosure relates to recombinant immunoglobulin endoglycosidases comprising an E mutation at position 263.

In certain embodiments, this disclosure contemplates recombinant immunoglobulin G-specific endoglycosidases disclosed herein as fusion constructs comprising an antibody heavy chain (Fc domain) and known mutations. As used herein, a “mutation,” “mutant,” or the like of an antibody heavy chain sequence refers to the expression of a variant amino acid(s) within a heavy chain antibody defined by positions compared to base amino acids within the sequence segment, e.g., of UNIPROTKB/SWISS-PROT having accession number P01857.1.

EPKSCDKTHTCPPCPAPELLGGPSVFLFPPKPKDTLMISRTPEVTCVVVDVSHEDP EVKFNWYVDGVEVHNAKTKPREEQYNSTYRVVSVLTVLHQDWLNGKEYKCKVSNKA LPAPIEKTISKAKGQPREPQVYTLPPSRDELTKNQVSLTCLVKGFYPSDIA VEWESNGQPE NNYKTTPPVLDSDGSFFLYSKLTVDKSRWQQGNVFSCSVMHEALHNHYTQKSLSLSPG K (SEQ ID NO: 55), wherein the N-terminal amino acid glutamic acid (E) is position 216.

The mutants may be constructed by building peptide sequences synthetically or, more typically, constructed using recombinant nucleic acid techniques, e.g., expression of the heavy chain in a cell from a template nucleic acid. Due to three codon translation of amino acids from nucleic acid, several three nucleotide codons may express the same amino acid variant. Sometimes the variant is due to a single nucleotide change, and sometimes the variant is due to more than one nucleotide change. Thus, reference to a “mutation,” “mutant,” or the like of a heavy chain antibody sequence are not necessarily limited to solely single nucleotide changes. In certain embodiments, the first or second heavy chain further has one, two, three, four, five, six, seven, eight, nine, ten, cleven, twelve, thirteen, fourteen, fifteen or more of the following mutations G236A, S239D, A330L, 1332E, S267E, L328F, P238D, H268F, S324T, S228P, G236R, L328R, L234A, L235A, M252Y, S254T, T256E, M428L, N434S, P329G, D265A, N297A, N297G, N297Q, F243L, R292P, Y300L, V305I, P396L, S298A, E333A, K334A, L234Y, L235Q, G236W, S239M, H268D, D270E, K326D, A330M, K334E, K326W, E333S, E345R, E430G, S440Y, L235E, N325S, wherein the mutation is in reference to positions in amino acid sequence (SEQ ID NO: 55) (segment of UNIPROTKB/SWISS-PROT: P01857.1), wherein the glutamic acid (E) is position 216.

As used herein, an “CP40,” refers to a protein produced by Corynebacterium pseudotuberculosis. CP40 is believed to be an endoglycosidase with putative activity on host glycoproteins and all known variants or substantial fragments thereof. One example has the amino acid sequence of:
MHNSPRSVSRLITVGITSALFASTFSAVASAESATLSKEPLKASPGRADTVGVQTT CNAKPIFFGYYRTWRDKAIQLKDDDPWKDKLQVKLTDIPEHVNMVSLFHVEDNQKSDQ QFWETFHREYQPELKKRGTRVVRTVGAQLLLNKIKDKNLYGKHVEDDYKYREIARDV YNEYVVKHNLDGLDVDMELRQVEKQLNLKWQLRKIMGAFSELMGPKAPANEGKKPD HEGYKYLIYDTFDNAQTSQVGLVADLVDYVLAQTYKKDTKESVTQVWNGFRDKINSC QFMAGYAHPEENDTNRFLTAVGEVNKSGAMQVAEWKPEGGEKGGTFAYALDRDGRT YDGDDFTTLKPTDFAFTKRAIELTTGESSTDLGKPTGSR (SEQ ID NO: 57) NCBI Reference Sequence: WP_013242749.1) wherein the N-terminal Methionine (M) is position one. Other examples include those designated by Accession numbers: WP_013242749.1, WP_058831578.1, WP_014522771.1, WP_058156943.1, WP_014733097.1, WP_138130220.1, WP_045421254.1, and WP_072577955.1.

In certain embodiments, this disclosure relates to a recombinant immunoglobulin G-specific endoglycosidase comprising the amino acid sequence of (CU43):
APAALSNAPLAASPGQADKVGAQATCAAKPIFFGYYRTWRDKAIELNDGDKWK DKLHTKLTDIPEQVDMVSLFHVPDNQKSDORFWETFDKEYHPTLKERGTKVVRTIGAK LLLNKIKEKGLYGQSREDDSKYREIAHEVYEEYVAKHNLDGLDVDMELREVEKYTNLR WQLRKIMGAFSELMGPKAPGNAGKKPGDDGYKYLIYDTFDNAQLAQVALVADVVDY VLAQTYDKGTEESITR VWNGFRDKINSCQFLAGYAHPEENDTNRFLTAIGDVDTSGAMN VAAWKPEGGEKGGTFAYALDRDGRTYDGDDLTTLKPTDFAFTKRAIELTKGISLTDLG (SEQ ID NO: 19) or variant thereof.

In certain embodiments, the recombinant immunoglobulin G-specific endoglycosidase comprises an FK peptide insertion at position 240 of CU43 or other peptide disclosed herein with similarity providing a peptide motif of the following amino acid sequence FFKD (SEQ ID NO: 50). In certain embodiments, the recombinant immunoglobulin G-specific endoglycosidase comprises a D to E amino acid change at amino acid position 263 providing a peptide motif of the following amino acid sequence TYEK (SEQ ID NO: 51). In certain embodiments, the recombinant immunoglobulin G-specific endoglycosidase or fusions comprise peptide motifs of the following amino acid sequences FFKD (SEQ ID NO: 50) and TYEK (SEQ ID NO: 51).

In certain embodiments, the recombinant immunoglobulin G-specific endoglycosidase comprises peptide motifs of the following amino acid sequences from an N-terminal to C-terminal order sequentially, from an N-terminal to C-terminal order sequentially, YYRTWRDK (SEQ ID NO: 1), TDIP, (SEQ ID NO: 2), LDGLDXIDME (SEQ ID NO: 3), FFKD (SEQ ID NO: 50), TYEK (SEQ ID NO: 51), SCQF (SEQ ID NO: 4), NRFL (SEQ ID NO: 5), and DRDG (SEQ ID NO: 6), wherein X¹ is I or V or any amino acid.

In certain embodiments, the recombinant immunoglobulin G-specific endoglycosidase does not contain an amino acid sequence KESV (SEQ ID NO: 48, contained in CP40) between TYEK (SEQ ID NO: 51) and SCQF (SEQ ID NO: 4).

In certain embodiments, the recombinant immunoglobulin G-specific endoglycosidase comprises peptide motifs of the following amino acid sequences from an N-terminal to C-terminal order sequentially, AXXXXXPLXXXXGXXXXXG (SEQ ID NO: 7), PIXXXYYRTWRDKXIXXXXXD (SEQ ID NO: 8), TDIPXXXXXXSLXHVXD, (SEQ ID NO: 9), QXSDXXFWXTFXXXYXPXLXXRGTXVXXTXXXXXXL (SEQ ID NO: 10), YXXXAXXXXXXYVXXHXLDGLDXDME, (SEQ ID NO: 11), LRXXMXAXXXLXGPXXXXN (SEQ ID NO: 12), LXYXTFFKDNAQXXXXXXV (SEQ ID NO: 56), AXXVXYVXXQTYEK (SEQ ID NO: 18), WNXXRXXXXSCQFXXXYAXPEEXDX (SEQ ID NO: 15), NRFLXAXGXGXVXXXXAXXXAXWXP (SEQ ID NO: 16), and GXKGGXXXYAXDRDGXTYDXXDXXTLXXTXFXXXKR (SEQ ID NO: 17), wherein X is individually and independently at each position any amino acid or variant thereof.

In certain embodiments, the recombinant immunoglobulin G-specific endoglycosidase comprises the amino acid sequence (CU43_ins240FK_D263E):
APAALSNAPLAASPGQADKVGAQATCAAKPIFFGYYRTWRDKAIELNDGDKWK DKLHTKLTDIPEQVDMVSLFHVPDNQKSDORFWETFDKEYHPTLKERGTKVVRTIGAK LLLNKIKEKGLYGQSREDDSKYREIAHEVYEEYVAKHNLDGLDVDMELREVEKYTNLR WQLRKIMGAFSELMGPKAPGNAGKKPGDDGYKYLIYDTFFKDNAQLAQVALVADVV DYVLAQTYEKGTEESITRVWNGFRDKINSCQFLAGYAHPEENDTNRFLTAIGDVDTSGA MNVAAWKPEGGEKGGTFAYALDRDGRTYDGDDLTTLKPTDFAFTKRAIELTKGISLTD LGTNGSRTPGANSSRTETSSFFNDPYYQKLMKEAGIADNTN (SEQ ID NO: 54), wherein the N-Terminal alanine (A) is position 32.

In certain embodiments, the recombinant immunoglobulin G-specific endoglycosidase comprises the amino acid sequence of (CU43_ins240FK_D263E with the signal sequence):
MRNVPQSVSRFATVGITSAIFAASLSTVASAAPAALSNAPLAASPGQADKVGA QATCAAKPIFFGYYRTWRDKAIELNDGDKWKDKLHTKLTDIPEQVDMVSLFHVPDNQK SDORFWETFDKEYHPTLKERGTKVVRTIGAKLLLNKIKEKGLYGQSREDDSKYREIAHE VYEEYVAKHNLDGLDVDMELREVEKYTNLRWQLRKIMGAFSELMGPKAPGNAGKKP GDDGYKYLIYDTFFKDNAQLAQVALVADVVDYVLAQTYEKGTEESITRVWNGFRDKI NSCQFLAGYAHPEENDTNRFLTAIGDVDTSGAMNVAAWKPEGGEKGGTFAYALDRDG RTYDGDDLTTLKPTDFAFTKRAIELTKGISLTDLGTNGSRTPGANSSRTETSSFFNDPYYQ KLMKEAGIADNTN (SEQ ID NO: 52) without the signal sequence or MRNVPQSVSRFATVGITSAIFAASLSTVASAAP (SEQ ID NO: 53) providing:
APAALSNAPLAASPGQADKVGAQATCAAKPIFFGYYRTWRDKAIELNDGDKWK DKLHTKLTDIPEQVDMVSLFHVPDNQKSDORFWETFDKEYHPTLKERGTKVVRTIGAK LLLNKIKEKGLYGQSREDDSKYREIAHEVYEEYVAKHNLDGLDVDMELREVEKYTNLR WQLRKIMGAFSELMGPKAPGNAGKKPGDDGYKYLIYDTFFKDNAQLAQVALVADVV DYVLAQTYEKGTEESITRVWNGFRDKINSCQFLAGYAHPEENDTNRFLTAIGDVDTSGA MNVAAWKPEGGEKGGTFAYALDRDGRTYDGDDLTTLKPTDFAFTKRAIELTKGISLTD LGTNGSRTPGANSSRTETSSFFNDPYYQKLMKEAGIADNTN (SEQ ID NO: 54).

In certain embodiments, this disclosure relates to recombinant immunoglobulin endoglycosidases comprising peptide motifs of the following amino acid sequences from an N-terminal to C-terminal order sequentially, YYRTWRDK (SEQ ID NO: 1), TDIP, (SEQ ID NO: 2), LDGLDX¹DME (SEQ ID NO: 3), SCQF (SEQ ID NO: 4), NRFL (SEQ ID NO: 5), and DRDG (SEQ ID NO: 6), wherein X¹ is I or V or any amino acid, or variant thereof, provided the immunoglobulin G-specific endoglycosidase does not contain an amino acid sequence KESV (SEQ ID NO: 48, contained in CP40) between LDGLDX¹DME (SEQ ID NO: 3) and SCQF (SEQ ID NO: 4).

In certain embodiments, this disclosure relates to recombinant immunoglobulin endoglycosidases comprise peptide motifs of the following amino acid sequences from an N-terminal to C-terminal order sequentially, AXXXXXPLXXXXGXXXXXG (SEQ ID NO: 7), PIXXXYYRTWRDKXIXXXXXD (SEQ ID NO: 8), TDIPXXXXXXSLXHVXD, (SEQ ID NO: 9), QXSDXXFWXTFXXXYXPXLXXRGTXVXXTXXXXXXL (SEQ ID NO: 10), YXXXAXXXXXXYVXXHXLDGLDXDME (SEQ ID NO: 11) LRXXMXAXXXLXGPXXXXN (SEQ ID NO: 12), LXYXTXXNAQXXXXXXV (SEQ ID NO: 13), AXXVXYVXXQTY (SEQ ID NO: 14), WNXXRXXXXSCQFXXXYAXPEEXDX (SEQ ID NO: 15), NRFLXAXGXGXVXXXXAXXXAXWXP (SEQ ID NO: 16), and GXKGGXXXYAXDRDGXTYDXXDXXTLXXTXFXXXKR (SEQ ID NO: 17), wherein X is individually and independently at each position any amino acid or variant thereof.

In certain embodiments, this disclosure relates to recombinant immunoglobulin endoglycosidases comprising peptide motifs of the following amino acid sequences from an N-terminal to C-terminal order sequentially, PVFF (SEQ ID NO: 22), YYRTWRDKAI (SEQ ID NO: 23), LTDIP (SEQ ID NO: 24), MVSLFHA (SEQ ID NO: 25), VRTT (SEQ ID NO: 26), HNLDGLDVDME (SEQ ID NO: 27), GPKA (SEQ ID NO: 28), LIYDTFD (SEQ ID NO: 29), VDYVL (SEQ ID NO: 30), SCQF (SEQ ID NO: 4), GYAHPEE (SEQ ID NO: 31), NRFETAIG (SEQ ID NO: 32), DRDGRTY (SEQ ID NO: 33), TFKR (SEQ ID NO: 34), or variant thereof.

In certain embodiments, this disclosure relates to recombinant immunoglobulin endoglycosidases comprising peptide motifs of the following amino acid sequences from an N-terminal to C-terminal order sequentially, PXXXXXSLXXXXGXXXXXG (SEQ ID NO: 35), PVFFXYYRTWRDKAIXXXXXD (SEQ ID NO: 36), LTDIPXXVXMVSLFHAXN (SEQ ID NO: 37), YXSDXXFWXTFXXXYXPXLKXRGTXVRTTXXAXXLL (SEQ ID NO: 38), DXXYREXAXXXXXXYVXXHNLDGLDVDME (SEQ ID NO: 39), WXIRKXMXAXSELXGPKAXXN (SEQ ID NO: 40), GYKXLIYDTFDXXXXXQ (SEQ ID NO: 41), AXXVDYVLXQTY (SEQ ID NO: 42), WNXXRXXXXSCQFXXGYAHPEEXDT (SEQ ID NO: 43), NRFETAIGXGXVXXSXAMXVAXWXP (SEQ ID NO: 44), and GGXKGGXFXYAXDRDGRTYXXDDXXXXKXTDFXTFKR (SEQ ID NO: 45), wherein X is individually and independently at each position any amino acid or variant thereof.

In certain embodiments, the recombinant immunoglobulin G-specific endoglycosidase comprises an FF peptide insertion at position 240 providing a peptide motif of the following amino acid sequence FFFD (SEQ ID NO: 20).

In certain embodiments, the recombinant immunoglobulin G-specific endoglycosidase comprises peptide motifs of the following amino acid sequences from an N-terminal to C-terminal order sequentially, YYRTWRDK (SEQ ID NO: 1), TDIP, (SEQ ID NO: 2), LDGLDXIDME (SEQ ID NO: 3), FFFD (SEQ ID NO: 20), SCQF (SEQ ID NO: 4), NRFL (SEQ ID NO: 5), and DRDG (SEQ ID NO: 6), wherein X¹ is I or V or any amino acid.

In certain embodiments, the recombinant immunoglobulin G-specific endoglycosidase comprises the amino acid sequence of:
AALSNAPLAASPGQADKVGAQATCAAKPIFFGYYRTWRDKAIELNDGDKWKDK LHTKLTDIPEQVDMVSLFHVPDNQKSDORFWETFDKEYHPTLKERGTKVVRTIGAKLLL NKIKEKGLYGQSREDDSKYREIAHEVYEEYVAKHNLDGLDVDMELREVEKYTNLRWQ LRKIMGAFSELMGPKAPGNAGKKPGDDGYKYLIYDTFFFDNAQLAQVALVADVVDYV LAQTYDKGTEESITR VWNGFRDKINSCQFLAGYAHPEENDTNRFLTAIGDVDTSGAMNV AAWKPEGGEKGGTFAYALDRDGRTYDGDDLTTLKPTDFAFTKRAIELTKGISLTDLGTN GSRTPGANSSRTETSSFFNDPYYQKLMKEAGIADNTN (SEQ ID NO: 46), wherein the N-Terminal alanine (A) is position 34.

In certain embodiments, the recombinant immunoglobulin G-specific endoglycosidase does not contain an amino acid sequence KESV (SEQ ID NO: 48, contained in CP40) between FFFD (SEQ ID NO: 20) and SCQF (SEQ ID NO: 4).

In certain embodiments, the recombinant immunoglobulin G-specific endoglycosidase comprises peptide motifs of the following amino acid sequences from an N-terminal to C-terminal order sequentially, AXXXXXPLXXXXGXXXXXG (SEQ ID NO: 7), PIXXXYYRTWRDKXIXXXXXD (SEQ ID NO: 8), TDIPXXXXXXSLXHVXD, (SEQ ID NO: 9), QXSDXXFWXTFXXXYXPXLXXRGTXVXXTXXXXXXL (SEQ ID NO: 10), YXXXAXXXXXXYVXXHXLDGLDXDME, (SEQ ID NO: 11), LRXXMXAXXXLXGPXXXXN (SEQ ID NO: 12), LXYXTFFFDNAQXXXXXXV (SEQ ID NO: 21), WNXXRXXXXSCQFXXXYAXPEEXDX (SEQ ID NO: 15), NRFLXAXGXGXVXXXXAXXXAXWXP (SEQ ID NO: 16), and GXKGGXXXYAXDRDGXTYDXXDXXTLXXTXFXXXKR (SEQ ID NO: 17), wherein X is individually and independently at each position any amino acid or variant thereof.

A “variant” refers to a polypeptide or polynucleotide that differs from a reference polypeptide or polynucleotide and retains essential properties. A typical variant of a polypeptide differs in amino acid sequence from another, reference polypeptide. Generally, differences are limited so that the sequences of the reference polypeptide and the variant are closely similar overall (homologous) and, in many regions, identical. A variant and reference polypeptide may differ in amino acid sequence by one or more modifications (e.g., substitutions, additions, and/or deletions). A substituted or inserted ammo acid residue may or may not be one encoded by the genetic code. A variant of a polypeptide may be naturally occurring such as an allelic variant, or it may be a variant that is not known to occur naturally.

Modifications and changes can be made in the structure of the peptides of this disclosure and still result in a molecule having similar characteristics as the peptide (e.g., a conservative amino acid substitution). For example, certain amino acids can be substituted for other amino acids in a sequence without appreciable loss of activity. Because it is the interactive capacity and nature of a peptide that defines the biological functional activity of the peptide, certain ammo acid sequence substitutions can be made in a peptide sequence and nevertheless obtain a peptide with like properties. Amino acid substitutions are generally based on the relative similarity of the ammo acid side-chain substituents, for example, their hydrophobicity, hydrophilicity, charge, size, and the like. Exemplary substitutions that take one or more of the foregoing characteristics into consideration are well known to those of skill in the art and include, but are not limited to (original residue: exemplary substitution): (Ala to Gly or Ser), (Arg to Lys), (Asn to Gln or His), (Asp to Glu, Cys, or Ser), (Gln to Asn), (Glu to Asp), (Gly to Ala), (His to Asn, Gln), (Leu to Ile or Val),

(Lys to Arg), (Met to Leu, Tyr), (Ser to Thr), (Thr to Ser), (Trp to Tyr), (Tyr to Trp or Phe), and (Val to Ile or Leu). Embodiments of this disclosure thus contemplate functional or biological equivalents of a peptide as set forth above. In general, homologous peptides of the present disclosure are characterized as having one or more amino acid substitutions, deletions, and/or additions.

“Identity,” as known in the art, is a relationship between two or more peptide sequences, as determined by comparing the sequences. In the art, “identity” also refers to the degree of sequence relatedness between peptides as determined by the match between strings of such sequences. “Identity” and “similarity” can be readily calculated by known methods. Preferred methods to determine identity are designed to give the largest match between the sequences tested.

In certain embodiments, sequence “identity” refers to the number of exactly matching amino acids (expressed as a percentage) in a sequence alignment between two sequences of the alignment calculated using the number of identical positions divided by the greater of the shortest sequence or the number of equivalent positions excluding overhangs wherein internal gaps are counted as an equivalent position.

The term “sample” is used in its broadest sense, in that it has chemical makeup that is physical for analysis, i.e., analyte. In one sense it can refer to a nasal fluid, saliva, cough droplets, or expelled droplets of saliva into the air, e.g., produced by speaking, or other lung fluid blood. In another sense, it is meant to include a specimen or culture obtained from any source, as well as biological and environmental samples. Biological samples include bodily fluids, urine, feces, nasal drip, seminal fluid, hair, skin (dead or epithelial layer of skin), finger or toenail clipping, and blood products such as plasma, serum, and the like. Preferably the sample is from a subject and encompass fluids, solids, tissues, and gases.

### Methods of Use

In certain embodiments, this disclosure relates to methods of cleaving or altering a glycan attached to immunoglobulin comprising contacting a glycosylated immunoglobulin and a recombinant immunoglobulin G-specific endoglycosidase or fusion as provided herein providing a glycan cleaved immunoglobulin. In certain embodiments, the immunoglobulin is in a sample from a subject, e.g., human patient.

### Pharmaceutical Compositions

In certain embodiments, this disclosure relates to pharmaceutical compositions comprising a recombinant immunoglobulin G-specific endoglycosidase or fusion as provided herein, nucleic acid, or vector as reported herein. The pharmaceutical compositions provided herein may generally include one or more pharmaceutically acceptable and/or approved carriers, additives, antibiotics, preservatives, diluents and/or stabilizers. Such auxiliary substances can be water, saline, glycerol, ethanol, wetting or emulsifying agents, pH buffering substances, or the like. Suitable carriers are typically large, slowly metabolized molecules such as proteins, polysaccharides, polylactic acids, polyglycolic acids, polymeric amino acids, amino acid copolymers, lipid aggregates, or the like.

In certain embodiments, the nucleic acid or recombinant vector encodes a recombinant disclosed herein having at least one open reading frame that can be translated by a cell or an organism provided with the nucleic acid, DNA, RNA, or mRNA. If more than one protein is translated, the proteins can be expressed in one vector/nucleic acid or in multiple (a plurality of) separate nucleic acids/vectors wherein the product of translation is a recombinant immunoglobulin G-specific endoglycosidase disclosed herein. The product may also be a fusion protein composed of more than one recombinant immunoglobulin endoglycosidase, e.g., a fusion protein that has two or more recombinant immunoglobulin endoglycosidases, wherein recombinant immunoglobulin endoglycosidases are optionally linked by self-cleaving linker sequences.

In certain embodiments, nucleic acid, DNA, RNA, or mRNA may be designed to have two (bicistronic) or more (multicistronic) open reading frames (ORF). An open reading frame in this context is a sequence including a start codon that can be used as a location to start translation of the encoded nucleic acid into a recombinant immunoglobulin G-specific endoglycosidase disclosed herein. Translation of such nucleic acid(s) yields two (bicistronic) or more (multicistronic) identical or distinct translation products/proteins (provided the ORFs are not identical). For expression in eukaryotes such nucleic acids may comprise an internal ribosomal entry site (IRES) sequence which allows for expression of two or more proteins on a single nucleic acid molecule.

In certain embodiments, the recombinant immunoglobulin endoglycosidase, nucleic acid, or recombinant vector may be administered naked without being associated with any further vehicle, e.g., mRNA, RNA, or DNA.

In certain embodiments, this disclosure relates to isolated blood products comprising or exposed to a recombinant immunoglobulin G-specific endoglycosidase or fusion as provided herein, or nucleic acid, vector, or attenuated E. coli strain encoding a recombinant immunoglobulin G-specific endoglycosidase or fusion as provided herein.

In certain embodiments, the blood product is plasma, whole blood, or platelets. In certain embodiments, the blood product contains cells isolated from human peripheral blood and stored for subsequent use including erythrocytes (red blood cells), leukocytes (white blood cells), and thrombocytes (platelets), packed red blood cell (PRBC) concentrate, platelet concentrate, fresh frozen plasma, or cryoprecipitate.

In certain embodiments, this disclosure relates to a blood product container, e.g., plastic bag with components for collecting and maintaining blood products, such as for whole blood, platletes, or plasma, typically comprising anticoagulants ACD (acid citrate dextrose), CPD (citrate phosphate dextrose) with or without adenine (ACD-A and CPD-A), or for apheresis comprising citric acid, sodium citrate, and dextrose in water (ACD).

In certain embodiments, the recombinant immunoglobulin G-specific endoglycosidase or fusion, nucleic acid, or recombinant vector may be administered in a pharmaceutical composition having a pharmaceutically acceptable excipient selected from lactose, sucrose, mannitol, tricthyl citrate, dextrose, cellulose, methyl cellulose, ethyl cellulose, hydroxyl propyl cellulose, hydroxypropyl methylcellulose, carboxymethylcellulose, croscarmellose sodium, polyvinyl N-pyrrolidone, crospovidone, ethyl cellulose, povidone, methyl and ethyl acrylate copolymer, polyethylene glycol, fatty acid esters of sorbitol, lauryl sulfate, gelatin, glycerin, glyceryl monooleate, silicon dioxide, titanium dioxide, talc, corn starch, carnauba wax, stearic acid, sorbic acid, magnesium stearate, calcium stearate, castor oil, mineral oil, calcium phosphate, starch, carboxymethyl ether of starch, iron oxide, triacetin, acacia gum, esters, or salts thereof.

In certain embodiments, the pharmaceutical composition is in the form of a sterilized pH buffered aqueous salt solution or a saline phosphate buffer between a pH of 6 to 8, optionally comprising a saccharide or polysaccharide.

In certain embodiment, the pharmaceutically acceptable excipient is a cationic or polycationic compound and/or with a polymeric carrier. In certain embodiments, the recombinant immunoglobulin G-specific endoglycosidase or fusion, nucleic acid, or recombinant vector are in a pharmaceutical composition associated with or complexed with a cationic or polycationic compound or a polymeric carrier. In certain embodiments, the cationic or polycationic compound is protamine, spermine, spermidine, poly-L-lysine (PLL), poly-histidine, or poly-arginine, cationic polysaccharides, such as chitosan, polybrene, cationic polymers, polyethyleneimine (PEI), homo-and co-polymers of lactic acid and glycolic acid, or polymethylmethacrylate.

Pharmaceutically acceptable carriers that may be used in these compositions include, but are not limited to, ion exchangers, alumina, aluminum stearate, lecithin, serum proteins, albumin, such as human serum albumin, buffer substances such as phosphates, glycine, sorbic acid, citric acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, hydrophilic polymers such as polyvinyl pyrrolidone, cellulose based substances, polyethylene glycol, sodium carboxymethylcellulose, polyacrylates, waxes, gelatin, polyethylene polyoxypropylene block polymers, polyethylene glycol and antioxidants including ascorbic acid and methionine; preservatives; low molecular weight (less than about 10 residues) polypeptides; proteins; and amino acids such as glycine, glutamine, asparagine, histidine, arginine, or lysine. In certain embodiments, the excipient may be one or more selected from the list consisting of NaCl, trehalose, sucrose, mannitol, and/or glycine.

The disclosure also contemplates products obtainable by further processing of a liquid formulation, such as a frozen, lyophilized or spray-dried product. Upon reconstitution, these solid products can become liquid formulations. In its broadest sense, therefore, the term “formulation” encompasses both liquid and solid formulations. However, solid formulations are understood as derivable from the liquid formulations (e.g. by freezing, frecze-drying or spray-drying), and hence have various characteristics that are defined by the features specified for liquid formulations hercin.

In certain embodiments, the formulations are isotonic in relation to human blood. Isotonic solutions possess the same osmotic pressure as blood plasma, and so can be intravenously infused into a subject without changing the osmotic pressure of the blood plasma.

In certain embodiments, the pharmaceutical composition is administered either systemically or locally, i.e., parenteral, subcutaneous, intravenous, intramuscular, intranasal, or any other path of administration. The mode of administration, the dose and the number of administrations can be optimized.

In certain embodiments, this disclosure relates to kits containing materials useful for aspects of the disclosure described herein as described above is provided. In certain embodiments, the kit comprises a container, a product label and a package insert. Suitable containers include, for example, bottles, vials, syringes, bags, and boxes containing the same. The containers may be of a variety of materials, e.g., glass, plastic, or cardboard. At least one active agent in the composition is a recombinant immunoglobulin G-specific endoglycosidase or fusion as provided herein, nucleic acid, or recombinant vector as disclosed herein. The product label on, or associated with, the container indicates the use of the composition. In certain embodiments, the kit may further comprise a second container comprising a pharmaceutically acceptable buffer, such as a phosphate buffer saline or a citrate buffered saline. It may further include other materials desirable from a user or commercial standpoint, including other buffers, diluents, filters, needles, and syringes. In certain embodiments, a dosage unit form can be, e.g., in the format of a prefilled syringe, an ampoule, cartridge or a vial.

In certain embodiments, this disclosure relates to kits or articles of manufacture, comprising a recombinant immunoglobulin G-specific endoglycosidase or fusion as provided herein, nucleic acid, or recombinant vector as disclosed herein and instructions for use by, e.g., a healthcare professional. The kits or articles of manufacture may include a container, vial, or a syringe containing the formulation as described herein. Preferably, the container, vial, or syringe is composed of glass, plastic, or a polymeric material. The syringe, ampoule, cartridge, or vial can be manufactured of any suitable material, such as glass or plastic and may include rubber materials, such as rubber stoppers for vials and rubber plungers and rubber seals for syringes and cartridges.

### Afucosylated Immunoglobulin G (IgG)-Specific Endoglycosidases

In humans, antibodies are produced by B cells and the most abundant type is immunoglobulin G (lgG). IgG antibodies protect against invading pathogens. N-linked glycans within the IgG Fc region are associated with IgG function and exist as variable composition in humans. Immune effector functions, such as antibody-dependent cellular cytotoxicity (ADCC), are modulated by the composition of the N-linked glycan at Asn297 of the Fc region of the antibody. Many circulating IgGs undergo fucosylation (approximately 94% in adults). Fucosylation is a post translational modification in the IgG Fc region where a fucose sugar conjugated at Asn297 is attached to the two N-linked complex-type oligosaccharides. This creates a steric hindrance and reduces their binding affinity FcyRIII. In contrast, removing the fructose sugar, i.e., afucosylated IgGs, which lack core fucose sugar units, circulate at low levels under homeostatic conditions (about 6% of total IgG) and have increased affinity for Fc γ receptors (FcγRIIIa and FcγRIIIb), present on the membrane of immune effector cells such as leukocytes or natural killer cells, leading to enhanced ADCC activity relative to those triggered by fucosylated IgG glycoforms. IgG Fc fucosylation in humans decreases slightly from birth to about 94% at adulthood, after which it remains fairly constant, albeit with a minor reduction throughout life.

In various infectious diseases, increased levels of antigen-specific afucosylated IgG1antibodies have been reported, and these correlate with different disease severities. Afucosylated IgG are specifically formed against enveloped viruses, such as severe acute respiratory syndrome coronavirus 2 (SARS-COV-2) and dengue virus (DENV) infections. Both viruses have been shown to induce a specific increase in antigen specific IgG1. Afucosylation and the levels of anti-DENV and anti-SARS-COV-2 afucosylated IgG1 were associated with progression from mild to more severe Dengue and COVID-19 diseases. This mediates stronger FcγRIIIa responses but also amplifies immune-mediated pathologies. Critically ill patients, but not those with mild symptoms, had high concentrations of afucosylated IgG antibodies against SARS-COV-2 and DENV, triggering proinflammatory cytokine release and acute phase responses, leading to cytokine storms.

For DENV and COVID-19, testing for levels of IgG1 glycoform levels from a sample, e.g., in the blood, provides an indicator of symptom severity in DENV and COVID-19 patients. The IgG1 fucosylation provides useful prognostic tool for the treatment of dengue patients with severe disease. Although antiviral antibodies generally confer protective functions, antibodies against dengue virus (DENV) are associated with enhanced disease susceptibility.

Antibodies can mediate DENV infection of leukocytes via Fc γ receptors, which may contribute to dengue disease pathogenesis. Antibody-dependent enhancement (ADE) in dengue is believed to be one of the major underlying mechanisms leading to increased severity in secondary DENV infection. ADE was shown to enhance viral entry into immune cells via their Fc γ receptors, which promotes viral replication, leading to increased viremia and pro-inflammatory responses. These contribute to disease pathologies including vascular hyperpermeability, a common cause of severe dengue. Afucosylated IgG1s have increased binding affinity to FcγRIIIa and FcγRIIIb; this may trigger higher uptake of DENV immune complexes and cause extensive inflammation, which are associated with ADE. Afucosylated IgG1s from dengue patients recognize the E proteins (viral envelope protein), and these antibody levels correlated with increased disease severity. See Wang et al. IgG antibodies to dengue enhanced for FcγRIIIA binding determine disease severity, Science, 2017, 355(6323):395-398. Having greater than 10% afucosylated anti-E IgG1 was reported to be a significant risk factor for thrombocytopenia and blocking of FcγRIIIa protected against platelet reduction in mice. Similarly, the presence of greater than 10% afucosylated maternal anti-E IgG1s predicted symptomatic primary dengue in infants.

Cytokine storm, also called hypercytokinemia, is a physiological reaction in which the innate immune system is hyperactivated and causes a life-threatening systemic inflammatory syndrome, consisting of an uncontrolled and excessive release of proinflammatory signaling molecules called cytokines that can be triggered by various therapies, pathogens, cancers, autoimmune conditions, and monogenic disorders. Normally, cytokines are part of the immune response to infection; however, sudden release in large quantities can cause multisystem organ failure and death.

Cytokine storms can be caused by a number of viral respiratory infections such as coronavirus, SARS-COV-1 and 2, influenza H1N1 and H5N1, Influenza B, Parainfluenza virus. Other causative agents include the Epstein-Barr virus, cytomegalovirus, group A streptococcus, and non-infectious conditions such as graft-versus-host disease. The viruses can invade lung epithelial cells and alveolar macrophages to produce viral nucleic acids which stimulates the infected cells to release cytokines and chemokines, activating macrophages, dendritic cells, and others.

Many people died from the coronavirus SARS-COV-2 due to COVID-19-induced hyper-inflammation with features of cytokine storm syndrome (CSS) and associated acute respiratory distress syndrome. It is contemplated that measuring or detecting biomarkers for fucosylated or afucosylated antibodies allows for guided treatment to prevention of cytokine storm or cytokine storm associated disorders, e.g., administering drugs to patients regardless of the underlying condition. Antibodies that were elicited by mRNA SARS-COV-2 vaccines were highly fucosylated and enriched in sialylation, both are modifications that reduce the inflammatory potential of IgG. Vaccine elicited IgG did not promote an inflammatory lung response. Thus, human IgG-FcyR interactions regulate inflammation in the lung and define distinct lung activities mediated by IgG antibodies that are associated with protection against, or progression to, severe COVID-19.

Specific afucosylated IgG responses have been identified against platelets and red blood cell (RBC) antigens. Similar afucosylated IgG were also found in various alloimmune responses, which are directed against surface-exposed, membrane-embedded proteins on RCB and platelets. RCB alloimmunization, or the formation of antibodies against non-self-antigens on RBCs, may occur after exposure to certain antigens through blood transfusion or pregnancy. Blood group antigens are diverse in structure, function, and immunogenicity. These antibodies may be leading to delayed hemolytic or serologic transfusion reactions or hemolytic disease of the fetus and newborn. Complications from RBC alloantibodies are a cause of transfusion-associated death.

Alloantibodies against red blood cells (RBCs) and platelets show low IgG-Fc fucosylation in patients, even down to 10% in several cases. Moreover, lowered IgG-Fc fucosylation is one of the factors that is used to determine disease severity in pregnancy-associated alloimmunizations, resulting in excessive thrombocytopenia and RBC destruction when targeted by afucosylated antibodies. RBC alloimmunization remains a major barrier to transfusion therapy in patients who have antibodies against multiple alloantigens. Modifying the glycosylation traits of proteins (in particular antibodies), tissues and cells provide for the development of glycan-based therapeutisc.

In this regard, Endoglycosidase S (EndoS) from Streptococcus pyogenes is an enzyme that efficiently deglycosylates the IgG Fc Complex-type N glycans indicating therapeutic potential against IgG-mediated disease by hydrolysis of IgG glycans following intravenous administration in rabbits and mice, resulting in modulation of IgG effector functions showing anti-inflammatory effects. Thus, influencing the glycosylation profiles of autoantigen specific IgGs represent an approach for intercepting pathogenic T cell and B cell responses.

### Recombinant Endo-β-N-acetylglucosaminidases

Endo-β-N-acetylglucosaminidases, hereafter referred to as endoglycosidases or GH18 enzymes, produced by various organisms catalyze the hydrolysis of N-linked glycans on glycoproteins. Most endoglycosidases recognize their glycoprotein substrates by glycan-specific, but protein-nonspecific, mechanisms. However, a subset of endoglycosidases specifically hydrolyzes the Asn297-linked glycan on IgG antibodies. This glycan is the major molecular determinant of Fc gamma receptor and complement Clq binding by IgG antibodies, interactions that in turn trigger antibody-mediated effector functions that are critical for the signaling properties of these antibodies. IgG-specific endoglycosidases are useful for the treatment or prevention of diseases or conditions mediated by IgG antibodies including, such as, autoimmunity and transplantation rejection. Many IgG-specific endoglycosidases are multi-domain proteins belonging to a family of enzymes exemplified by EndoS and EndoS2, which are secreted by various strains of Streptococcus pyogenes. A single-domain IgG-specific endoglycosidase, CP40, produced by Corynebacterium pseudotuberculosis has also been identified.

Reported herein is the identification and validation of additional members of a single-domain IgG specific endoglycosidase family. To identify putative homologs to the single-domain IgG-specific endoglycosidase CP40, sequence similarity networks (SSN) were constructed using all annotated GH18 enzymes from the CAZY database and the Enzyme Similarity Tool (EFI-EST). Cytoscape™M software was used for deep analysis of the resulting SSNs. By selecting an alignment score threshold of 50 (to reach an alignment stringency that allowed separation into distinct clusters in which proteins shared sequence identity lower than 35%), CP40 from C. pseudotuberculosis was located in a cluster containing 120 additional GH18 enzymes. Also located within this cluster were the multi-domain IgG-specific endoglycosidades, EndoS and EndoS2.

Sequences were filtered these based on sequence length, sequence identity, and alpha-fold model structure prediction. Of the 120 total GH enzymes, 48 were composed of single polypeptide sequences of greater than 600 amino acids, indicating the presence of multiple domains that may be involved in the recognition and/or binding of glycoprotein substrates. ORFs containing less than 300 residues were considered incompletes and discarded for the analysis. The remaining putative single-domain ORFs were subject to comprehensive comparison of their predicted three-dimensional structures using AlphaFold™. From this analysis, four homologs to CP40, were identified and characterized by sequence identities of 40-90% and a highly conserved fold, specifically in certain regions around the active site that appear as short helixes in CP40 and these four CP40 homologs (or CP40-like enzymes).

To increase sample size, another SSN was performed using Glyco_hydro_18 (PF00704) from the Pfam database, which contains 79,058 sequences of glycoside hydrolases from the GH18 family. By selecting an alignment score threshold of 60 (to reach a 35-40% sequence identity between the sequences in each cluster), CP40 from C. pseudotuberculosis was located within a cluster containing another 160 proteins. Three subclusters were identified, including those: (I) containing multi-domain IgG-specific endoglycosidases including EndoS, EndoS2 and other EndoS/S2-like proteins; (II) containing a single GH18 domain alone or paired with additional glycoside hydrolase domains, such as EndoCOM and EndoE (containing GH18 and GH20domains); and (III) containing CP40 and its close homologs. Of these CP40 homologs, a strain of C. pseudotuberculosis (strain 258) shares 91% amino acidic sequence identity with CP40. In the interface between sub-clusters II and III, CP40-like proteins were found from a genus of actinobacteria different from Corynebacteria, identified as Winkia. Hidden-Markov analysis of

CP40 sequence using HMMER also identified this hit. Winkia neuii BV029A5 or Actinomyces neuii were identified in the top 3 hits (e value 3.le-55), indicating a highly similar sequence pattern with CP40 protein. Also using HMM, it was found that CP40 shares high similarity with C. mustalea (e-value_1.9e-63), although this protein contains a triple helix bundle and a long flexible linker between the GH18 domain and a C-terminal transmembrane domain. Additionally, after BLAST searching CP40-like genes against non-redundant NCBI databases, two additional putative CP40-like genes from C. silvaticum and C. belfantii were found that were not in the Uniprot, Pfam and CAZY databases.

Multiple sequence alignments of CP40 and its closest homologs from Corynebacterium species was performed identified in the SSN analysis. DonE, an endoglycosidase from Bacteroides fragilis, was included in the sequence alignment as a negative control, as it has been reported to hydrolyze complex type (CT) N-glycans from transferrin but is unable to deglycosylate IgG or IgA. The GH18 domain sequence of EndoS2, which, by itself, also exhibits IgG-specific ENGase activity without the requirement of additional domains to specifically deglycosylate IgG was also included. At least 23 residues that are 100% conserved in all CP40-like proteins and the EndoS2 GH18 domain that are, additionally, different from those in DonE were identified. All CP40-like proteins contained two highly conserved cysteine residues (Cys57 and Cys284, numeration in CP40) that form a disulfide bond in AlphaFold™ modeled structures. Multiple sequence alignments were performed using CP40 and the four CP40-like enzymes identified by the methods described above. These enzymes have a sequence identity with CP40 as high as 91% and as low as 38%.

To define the enzymatic functionality of CP40 and the CP40-like enzymes, the ORFs of these four CP40 orthologs were cloned into the pET28a vector between BamHI-Xhol sites, containing His6 tag on the N-terminus. Codon optimization based on E. coli codon-usage was performed for all sequences. The signal peptide of each genetic construct was removed based on Signal P 6.0 prediction. the transmembrane domain of one homolog (named as CM49) based on hydrophobicity prediction using the Phobius server was deleted. The long flexible linker of CM49 was also removed based on its AlphaFold™ model prediction, but a C-terminal triple helix bundle was maintained (residues 339-394). One can express these proteins in E. coli and purify them to homogeneity by affinity chromatography.

### Generation of Afucosylated-IgG1-Specific Endoglycosidase

Single-domain endoglycosidases were identified from bacterial Corynebacterium species that specifically catalyzes the hydrolysis of the beta-1,4 linkage between the first two N-acetylglucosamine residues of the complex-type N-linked glycan located on the Asn297 residue of the Fc region of host IgG antibodies. These examples are contemplated to prevent interactions between IgGs and Fc γ receptors and the ability of the antibodies to activate the complement pathway during infection.

A strategy was devised to identify endoglycosidases that are specific and selective for afucosylated IgGs. The X-ray crystal and cryo-EM structures of the IgG-specific endoglycosidase EndoS in complex with its substrate IgG-CT (containing a GOF N-glycan on Fc Asn297) was evaluated. The X-ray crystal structures of one inactive mutant of a single-domain IgG-specific endoglycosidase from Corynebacterium ulcerans (CU43) was solved at 2.3 A resolution (pdb code: 8UEN) and 2.6 A resolution (pdb code: 8URA) and in complex with its substrate IgG-CT (E382S) at 3.62 Å resolution (pdb code: 8URO). The crystal structure of CU43 containing the N-glycan was used as a reference model/template to dock the GOF glycan (biantennary N-glycan that contains terminal N-acetylglucosamine residues, and fucosylation on the core GlcNAc) into the CU43 crystal structure, which allows identification of interaction with the fucose binding pocket on CU43.

Different Alphafold2™ models of enzyme mutants were generated and considered positive hits (around 20% of these) because they exhibited significant clashes with the fucose moiety. Recombinant variant CU43 enzymes were expressed, purified and experimentally tested for their catalytic activity and selectivity using afucosylated and fucosylated glycoforms of IgG1 antibodies (recombinantly produced in Expi293cells in the presence or absence of the fucose inhibitor 2-deoxy-2-fluoro-Lfucose). Different variants of CU43 were identified with high selectivity for afucosylated IgG1.

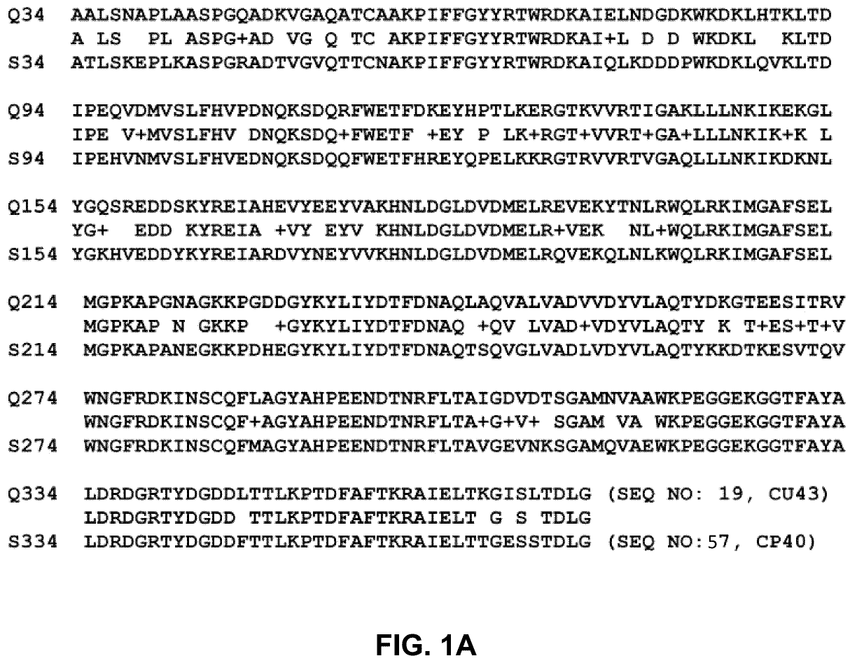

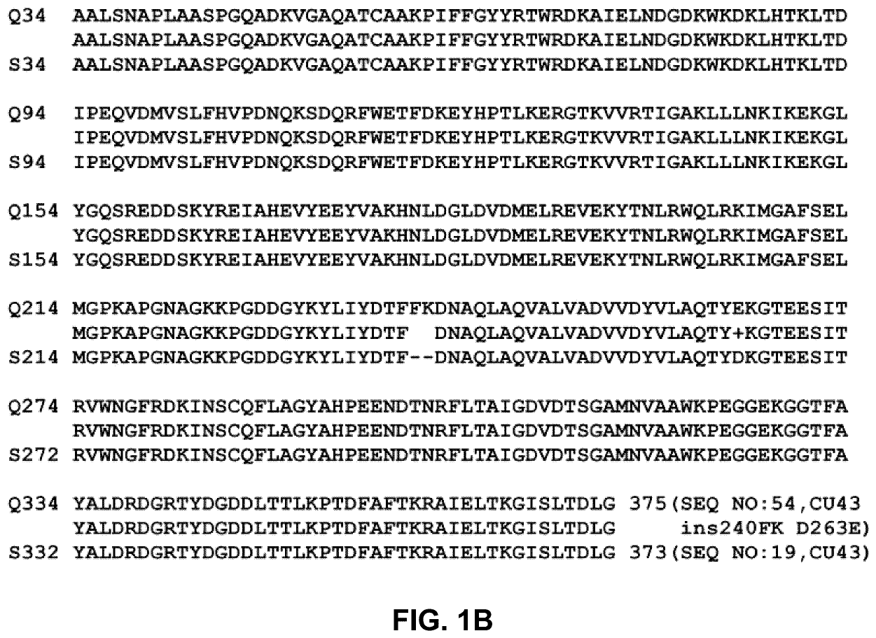

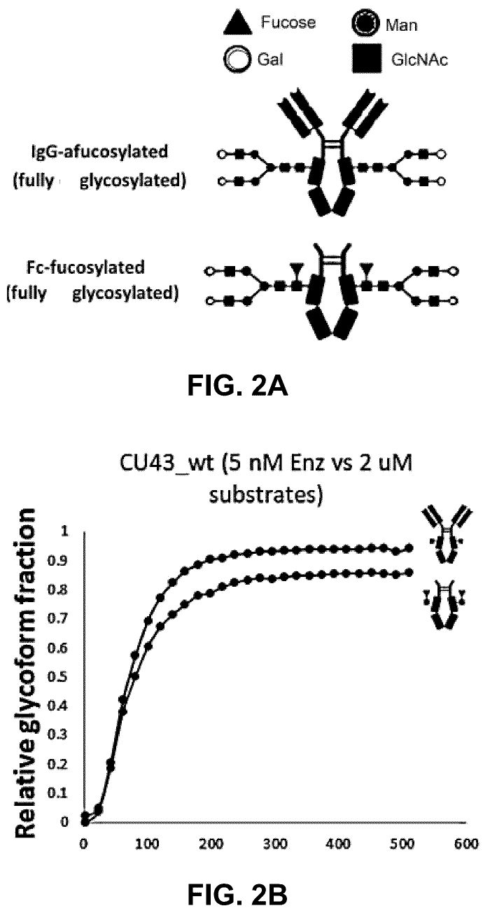

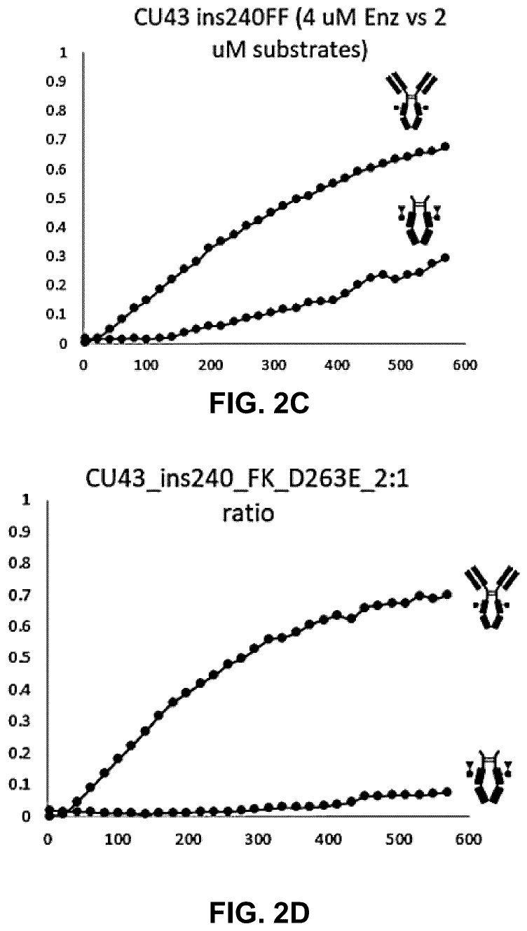

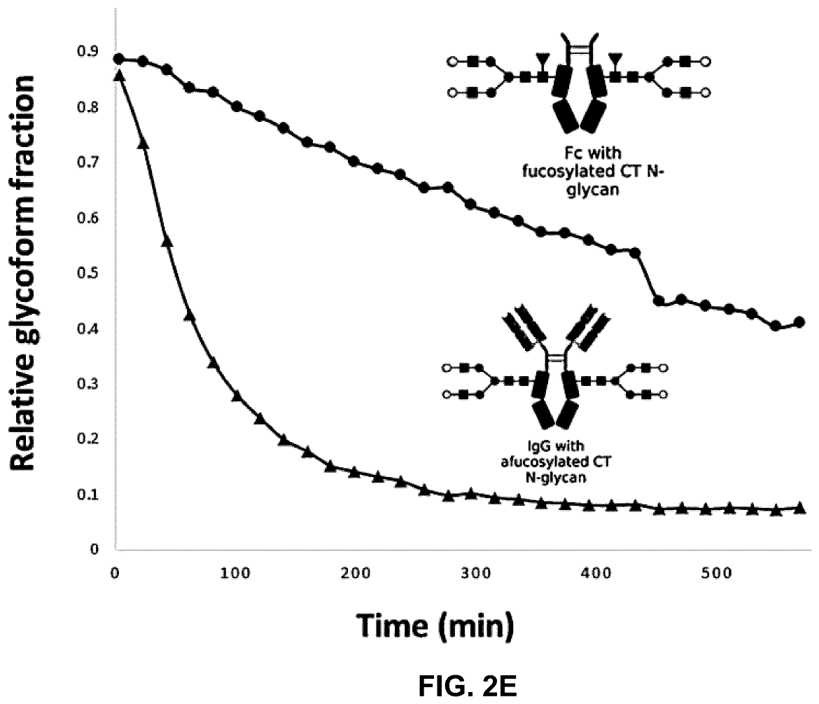

FIG. 1A shows a sequence comparison of CU43 (SEQ ID NO: 19, Q1) and CP40 (SEQ ID NO: 57, S1). FIG. 1B shows a sequence comparison of CU43_ins240FK_D263E (SEQ ID NO: 54, Query, Q) and CU43 (SEQ ID NO: 19, Subject, S). FIG. 2A illustrates the glycan structures of an antibody and Fc with fucosylated (with triangle fucose) and afucosylated (without triangle) modifications at Asn297. FIG. 2B shows data on the relative changes in deglycosylation over time when exposed immunoglobulin G (IgG) fucosylated or afucosylated to CU43 which shows similar preference for fucosylated and afucosylated Fc-IgG1. FIG. 2C shows data on the relative changes in deglycosylation over time when exposed to immunoglobulin G (IgG) fucosylated or afucosylated to CU43 with an FF insertion at position 240 (CU43_ins240FF). Different variants of CU43 were identified with high selectivity for afucosylated IgG1. Variant CU43_ins240FK_D263E exhibited improved preference (around 93% preference for afucosylated vs fucosylated CT-N-glycans) (FIG. 2D). After consumption of the IgG afucosylated diglycosylated species, approximately 70% of the completely glycosylated Fc-fucosylated intact species remained (FIG. 2E).

Informal Sequence Listing
YYRTWRDK Peptide Motif (SEQ ID NO: 1):
YYRTWRDK
TDIP Peptide Motif (SEQ ID NO: 2):
TDIP
LDGLDX1DME Peptide Motif (SEQ ID NO: 3;
wherein X1 is I or V or any amino acid):
LDGLDX1DME
SCQF Peptide Motif (SEQ ID NO: 4):
SCQF
NRFL Peptide Motif (SEQ ID NO: 5):
NRFL
DRDG Peptide Motif (SEQ ID NO: 6):
DRDG
AXXXXXPLXXXXGXXXXXG Peptide Motif (SEQ ID NO: 7; wherein X is individually and
independently at each position any amino acid or variant thereof):
AXXXXXPLXXXXGXXXXXG
PIXXXYYRTWRDKXIXXXXXD Peptide Motif (SEQ ID NO: 8; wherein X is individually and
independently at each position any amino acid or variant thereof):
PIXXXYYRTWRDKXIXXXXXD
TDIPXXXXXXSLXHVXD Peptide Motif (SEQ ID NO: 9; wherein X is individually and
independently at each position any amino acid or variant thereof):
TDIPXXXXXXSLXHVXD
QXSDXXFWXTFXXXYXPXLXXRGTXVXXTXXXXXXL Peptide Motif (SEQ ID NO: 10;
wherein X is individually and independently at each position is any amino
acid or variant thereof):
QXSDXXFWXTFXXXYXPXLXXRGTXVXXTXXXXXXL
YXXXAXXXXXXYVXXHXLDGLDXDME Peptide Motif (SEQ ID NO: 11; wherein X is
individually and independently at each position any amino acid or variant thereof):
YXXXAXXXXXXYVXXHXLDGLDXDME
LRXXMXAXXXLXGPXXXXN Peptide Motif (SEQ ID NO: 12 wherein X is individually and
independently at each position any amino acid or variant thereof):
LRXXMXAXXXLXGPXXXXN
LXYXTXXNAQXXXXXXV (SEQ ID NO: 13; wherein X is individually and independently at
each position any amino acid or variant thereof),
LXYXTXXNAQXXXXXXV
AXXVXYVXXQTY (SEQ ID NO: 14; wherein X is individually and independently at each
position any amino acid or variant thereof),
AXXVXYVXXQTY
WNXXRXXXXSCQFXXXYAXPEEXDX (SEQ ID NO: 15; wherein X is individually and
independently at each position any amino acid or variant thereof):
WNXXRXXXXSCQFXXXYAXPEEXDX
NRFLXAXGXGXVXXXXAXXXAXWXP (SEQ ID NO: 16; wherein X is individually and
independently at each position any amino acid or variant thereof):
NRFLXAXGXGXVXXXXAXXXAXWXP
GXKGGXXXYAXDRDGXTYDXXDXXTLXXTXFXXXKR (SEQ ID NO: 17; wherein X is
individually and independently at each position any amino acid or variant thereof):
GXKGGXXXYAXDRDGXTYDXXDXXTLXXTXFXXXKR
AXXVXYVXXQTYEK (SEQ ID NO: 18; wherein X is individually and independently at each
position any amino acid or variant thereof)
AXXVXYVXXQTYEK
CU43 Sequence (SEQ ID NO: 19)
APAALSNAPLAASPGQADKVGAQATCAAKPIFFGYYRTWRDKAIELNDGDKWKDKLHT
KLTDIPEQVDMVSLFHVPDNQKSDQRFWETFDKEYHPTLKERGTKVVRTIGAKLLLNK
IKEKGLYGQSREDDSKYREIAHEVYEEYVAKHNLDGLDVDMELREVEKYTNLRWQLR
KIMGAFSELMGPKAPGNAGKKPGDDGYKYLIYDTFDNAQLAQVALVADVVDYVLAQT
YDKGTEESITRVWNGFRDKINSCQFLAGYAHPEENDTNRFLTAIGDVDTSGAMNVAA
WKPEGGEKGGTFAYALDRDGRTYDGDDLTTLKPTDFAFTKRAIELTKGISLTDLG
FFFD Peptide Motif (SEQ ID NO: 20):
FFFD
LXYXTFFFDNAQXXXXXXV Peptide Motif (SEQ ID NO: 21; wherein X is individually and
independently at each position any amino acid or variant thereof):
LXYXTFFFDNAQXXXXXXV
PVFF Peptide Motif (SEQ ID NO: 22):
PVFF
YYRTWRDKAI Peptide Motif (SEQ ID NO: 23):
YYRTWRDKAI
LTDIP Peptide Motif (SEQ ID NO: 24):
LTDIP
MVSLFHA Peptide Motif (SEQ ID NO: 25):
MVSLFHA
VRTT Peptide Motif (SEQ ID NO: 26):
VRTT
HNLDGLDVDME Peptide Motif (SEQ ID NO: 27):
HNLDGLDVDME
GPKA Peptide Motif (SEQ ID NO: 28):
GPKA
LIYDTFD Peptide Motif (SEQ ID NO: 29):
LIYDTFD
VDYVL Peptide Motif (SEQ ID NO: 30):
VDYVL
GYAHPEE Peptide Motif (SEQ ID NO: 31):
GYAHPEE
NRFETAIG Peptide Motif (SEQ ID NO: 32):
NRFETAIG
DRDGRTY Peptide Motif (SEQ ID NO: 33):
DRDGRTY
TFKR Peptide Motif (SEQ ID NO: 34):
TFKR
PXXXXXSLXXXXGXXXXXG Peptide Motif (SEQ ID NO: 35; wherein X is individually and
independently at each position any amino acid or variant thereof):
PXXXXXSLXXXXGXXXXXG
PVFFXYYRTWRDKAIXXXXXD Peptide Motif (SEQ ID NO: 36; wherein X is individually and
independently at each position any amino acid or variant thereof):
PVFFXYYRTWRDKAIXXXXXD
LTDIPXXVXMVSLFHAXN Peptide Motif (SEQ ID NO: 37; wherein X is individually and
independently at each position any amino acid or variant thereof):
LTDIPXXVXMVSLFHAXN
YXSDXXFWXTFXXXYXPXLKXRGTXVRTTXXAXXLL Peptide Motif (SEQ ID NO: 38;
wherein X is individually and independently at each position any
amino acid or variant thereof):
YXSDXXFWXTFXXXYXPXLKXRGTXVRTTXXAXXLL
DXXYREXAXXXXXXYVXXHNLDGLDVDME Peptide Motif (SEQ ID NO: 39; wherein X is
individually and independently at each position any amino acid or variant thereof):
DXXYREXAXXXXXXYVXXHNLDGLDVDME
WXIRKXMXAXSELXGPKAXXN Peptide Motif (SEQ ID NO: 40; wherein X is individually
and independently at each position any amino acid or variant thereof):
WXIRKXMXAXSELXGPKAXXN
GYKXLIYDTFDXXXXXQ Peptide Motif (SEQ ID NO: 41; wherein X is individually and
independently at each position any amino acid or variant thereof):
GYKXLIYDTFDXXXXXQ
AXXVDYVLXQTY Peptide Motif (SEQ ID NO: 42; wherein X is individually and independently
at each position any amino acid or variant thereof):
AXXVDYVLXQTY
WNXXRXXXXSCQFXXGYAHPEEXDT Peptide Motif (SEQ ID NO: 43; wherein X is
individually and independently at each position any amino acid or variant thereof):
WNXXRXXXXSCQFXXGYAHPEEXDT
NRFETAIGXGXVXXSXAMXVAXWXP Peptide Motif (SEQ ID NO: 44; wherein X is
individually and independently at each position any amino acid or variant thereof):
NRFETAIGXGXVXXSXAMXVAXWXP
GGXKGGXFXYAXDRDGRTYXXDDXXXXKXTDFXTFKR Peptide Motif (SEQ ID NO: 45;
wherein X is individually and independently at each position any
amino acid or variant thereof):
GGXKGGXFXYAXDRDGRTYXXDDXXXXKXTDFXTFKR
Recombinant immunoglobulin G-specific endoglycosidase (SEQ ID NO: 46; wherein the N-
Terminal alanine (A) is position 34):
AALSNAPLAASPGQADKVGAQATCAAKPIFFGYYRTWRDKAIELNDGDKWKDKLHTK
LTDIPEQVDMVSLFHVPDNQKSDQRFWETFDKEYHPTLKERGTKVVRTIGAKLLLNKIK
EKGLYGQSREDDSKYREIAHEVYEEYVAKHNLDGLDVDMELREVEKYTNLRWQLRKI
MGAFSELMGPKAPGNAGKKPGDDGYKYLIYDTFFFDNAQLAQVALVADVVDYVLAQT
YDKGTEESITRVWNGFRDKINSCQFLAGYAHPEENDTNRFLTAIGDVDTSGAMNVAAW
KPEGGEKGGTFAYALDRDGRTYDGDDLTTLKPTDFAFTKRAIELTKGISLTDLGTNGSR
TPGANSSRTETSSFFNDPYYQKLMKEAGIADNTN
Recombinant immunoglobulin G-specific endoglycosidase (SEQ ID NO: 47):
MRNVPQSVSRFATVGITSAIFAASLSTVASAAPAALSNAPLAASPGQADKVGAQATCA
AKPIFFGYYRTWRDKAIELNDGDKWKDKLHTKLTDIPEQVDMVSLFHVPDNQKSDQRF
WETFDKEYHPTLKERGTKVVRTIGAKLLLNKIKEKGLYGQSREDDSKYREIAHEVYEEY
VAKHNLDGLDVDMELREVEKYTNLRWQLRKIMGAFSELMGPKAPGNAGKKPGDDGY
KYLIYDTFFKDNAQLAQVALVADVVDYVLAQTYEKGTEESITRVWNGFRDKINSCQFL
AGYAHPEENDTNRFLTAIGDVDTSGAMNVAAWKPEGGEKGGTFAYALDRDGRTYDGD
DLTTLKPTDFAFTKRAIELTKGISLTDLGTNGSRTPGANSSRTETSSFFNDPYYQKLMKE
AGIADNTN
KESV CP40 Section (SEQ ID NO: 48):
KESV
Signal sequence (SEQ ID NO: 49)
MRNVPQSVSRFATVGITSAIFAASLSTVASA
FFKD Peptide Motif (SEQ ID NO: 50):
FFKD
TYEK Peptide Motif (SEQ ID NO: 51)
TYEK
Recombinant immunoglobulin G-specific endoglycosidase CU43_ins240FK_D263E
with the signal sequence (SEQ ID NO: 52):
MRNVPQSVSRFATVGITSAIFAASLSTVASAAPAALSNAPLAASPGQADKVGAQATCA
AKPIFFGYYRTWRDKAIELNDGDKWKDKLHTKLTDIPEQVDMVSLFHVPDNQKSDQRF
WETFDKEYHPTLKERGTKVVRTIGAKLLLNKIKEKGLYGQSREDDSKYREIAHEVYEEY
VAKHNLDGLDVDMELREVEKYTNLRWQLRKIMGAFSELMGPKAPGNAGKKPGDDGY
KYLIYDTFFKDNAQLAQVALVADVVDYVLAQTYEKGTEESITRVWNGFRDKINSCQFL
AGYAHPEENDTNRFLTAIGDVDTSGAMNVAAWKPEGGEKGGTFAYALDRDGRTYDGD
DLTTLKPTDFAFTKRAIELTKGISLTDLGTNGSRTPGANSSRTETSSFFNDPYYQKLMKE
AGIADNTN
Signal Sequence (SEQ ID NO: 53):
MRNVPQSVSRFATVGITSAIFAASLSTVASAAP
Recombinant immunoglobulin G-specific endoglycosidase CU43_ins240FK_D263E
(SEQ ID NO: 54; wherein the N-Terminal alanine (A) is position 32):
APAALSNAPLAASPGQADKVGAQATCAAKPIFFGYYRTWRDKAIELNDGDKWKDKLHT
KLTDIPEQVDMVSLFHVPDNQKSDQRFWETFDKEYHPTLKERGTKVVRTIGAKLLLNK
IKEKGLYGQSREDDSKYREIAHEVYEEYVAKHNLDGLDVDMELREVEKYTNLRWQLR
KIMGAFSELMGPKAPGNAGKKPGDDGYKYLIYDTFFKDNAQLAQVALVADVVDYVLA
QTYEKGTEESITRVWNGFRDKINSCQFLAGYAHPEENDTNRFLTAIGDVDTSGAMNV
AAWKPEGGEKGGTFAYALDRDGRTYDGDDLTTLKPTDFAFTKRAIELTKGISLTDLG
TNGSRTPGANSSRTETSSFFNDPYYQKLMKEAGIADNTN
Segment of UNIPROTKB/SWISS-PROT: P01857.1 (SEQ ID NO: 55):
EPKSCDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAP
IEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSPGK
LXYXTFFKDNAQXXXXXXV Peptide Motif (SEQ ID NO: 56, wherein X is individually and
independently at each position any amino acid or variant thereof):
LXYXTFFKDNAQXXXXXXV
CP40 Amino Acid Sequence (SEQ ID NO: 57; NCBI Reference Sequence: WP_013242749.1):
ATLSKEPLKASPGRADTVGVQTTCNAKPIFFGYYRTWRDKAIQLKDDDPWKDKLQVKL
TDIPEHVNMVSLFHVEDNQKSDQQFWETFHREYQPELKKRGTRVVRTVGAQLLLNKI
KDKNLYGKHVEDDYKYREIARDVYNEYVVKHNLDGLDVDMELRQVEKQLNLKWQLRK
IMGAFSELMGPKAPANEGKKPDHEGYKYLIYDTFDNAQTSQVGLVADLVDYVLAQTY
KKDTKESVTQVWNGFRDKINSCQFMAGYAHPEENDTNRFLTAVGEVNKSGAMQVAEW
KPEGGEKGGTFAYALDRDGRTYDGDDFTTLKPTDFAFTKRAIELTTGESSTDLGKPTG
SR

What is claimed is:

1. A recombinant immunoglobulin G-specific endoglycosidase comprising a dipeptide FK insertion at position at position 240 wherein the dipeptide FK insertion is in reference to positions in SEQ ID NO: 57, wherein the N-terminal amino acid alanine (A) is position 34.

2. The recombinant immunoglobulin G-specific endoglycosidase of claim 1 comprising a E mutation at position 263.

3. The recombinant immunoglobulin G-specific endoglycosidase of claim 1 comprising peptide motifs of the following amino acid sequences FFKD (SEQ ID NO: 50) and TYEK (SEQ ID NO: 51).

4. The recombinant immunoglobulin G-specific endoglycosidase of claim 3 comprising peptide motifs of the following amino acid sequences from an N-terminal to C-terminal order sequentially, from an N-terminal to C-terminal order sequentially, YYRTWRDK (SEQ ID NO: 1), TDIP, (SEQ ID NO: 2), LDGLDX¹DME (SEQ ID NO: 3), FFKD (SEQ ID NO: 50), TYEK (SEQ ID NO: 51), SCQF (SEQ ID NO: 4), NRFL (SEQ ID NO: 5), and DRDG (SEQ ID NO: 6), wherein X¹ is I or V or any amino acid.

5. The recombinant immunoglobulin G-specific endoglycosidase of claim 3 which does not contain an amino acid sequence KESV (SEQ ID NO: 48, contained in CP40) between TYEK (SEQ ID NO: 51) and SCQF (SEQ ID NO: 4).

6. The recombinant immunoglobulin G-specific endoglycosidase of claim 3 comprising peptide motifs of the following amino acid sequences from an N-terminal to C-terminal order sequentially:
AXXXXXPLXXXXGXXXXXG (SEQ ID NO: 7),
PIXXXYYRTWRDKXIXXXXXD (SEQ ID NO: 8),
TDIPXXXXXXSLXHVXD, (SEQ ID NO: 9),
QXSDXXFWXTFXXXYXPXLXXRGTXVXXTXXXXXXL (SEQ ID NO: 10),
YXXXAXXXXXXYVXXHXLDGLDXDME, (SEQ ID NO: 11),
LRXXMXAXXXLXGPXXXXN (SEQ ID NO: 12),
LXYXTFFKDNAQXXXXXXV (SEQ ID NO: 56),
AXXVXYVXXXTYEK (SEQ ID NO: 18),
WNXXRXXXXSCQFXXXYAXPEEXDX (SEQ ID NO: 15),
NRFLXAXGXGXVXXXXAXXXAXWXP (SEQ ID NO: 16), and
GXKGGXXXYAXDRDGXTYDXXDXXTLXXTXFXXXKR (SEQ ID NO: 17), wherein X is individually and independently at each position is any amino acid.

7. The recombinant immunoglobulin G-specific endoglycosidase of claim 3 comprising the amino acid sequence (CU43_ins240FK_D263E)
(SEQ ID NO: 54)
APAALSNAPLAASPGQADKVGAQATCAAKPIFFGYYRTWRDKAIE
LNDGDKWKDKLHTKLTDIPEQVDMVSLFHVPDNQKSDQRFWETFD
KEYHPTLKERGTKVVRTIGAKLLLNKIKEKGLYGQSREDDSKYRE
IAHEVYEEYVAKHNLDGLDVDMELREVEKYTNLRWQLRKIMGAFS
ELMGPKAPGNAGKKPGDDGYKYLIYDTFFKDNAQLAQVALVADVV
DYVLAQTYEKGTEESITRVWNGFRDKINSCQFLAGYAHPEENDTN
RFLTAIGDVDTSGAMNVAAWKPEGGEKGGTFAYALDRDGRTYDGD
DLTTLKPTDFAFTKRAIELTKGISLTDLGTNGSRTPGANSSRTET
SSFFNDPYYQKLMKEAGIADNTN.

8. A fusion protein comprising any of the immunoglobulin endoglycosidases as provided in claim 1.

9. The fusion protein of claim 8, comprising an immunoglobulin Fc domain.

10. A nucleic acid encoding a recombinant immunoglobulin G-specific endoglycosidase as provided in claim 1 in operable combination with a heterologous promoter.

11. The nucleic acid of claim 10 which is mRNA, RNA, or DNA.

12. A live attenuated E. coli strain comprising a recombinant immunoglobulin G-specific endoglycosidase as provided in claim 1.

13. A vector comprising a nucleic acid encoding the fusion protein of claim 8.


SEQUENCE LISTING (57 sequences)

SEQ ID NO: 1 - protein, 8 aa, synthetic construct
YYRTWRDK

SEQ ID NO: 2 - protein, 4 aa, synthetic construct
TDIP

SEQ ID NO: 3 - protein, 9 aa, synthetic construct
  VARIANT 6: note = X = Ile or Val
LDGLDXDME

SEQ ID NO: 4 - protein, 4 aa, synthetic construct
SCQF

SEQ ID NO: 5 - protein, 4 aa, synthetic construct
NRFL

SEQ ID NO: 6 - protein, 4 aa, synthetic construct
DRDG

SEQ ID NO: 7 - protein, 19 aa, synthetic construct
  VARIANT 2: note = X is any amino acid or variant thereof
  VARIANT 3: note = X is any amino acid or variant thereof
  VARIANT 4: note = X is any amino acid or variant thereof
  VARIANT 5: note = X is any amino acid or variant thereof
  VARIANT 6: note = X is any amino acid or variant thereof
  VARIANT 9: note = X is any amino acid or variant thereof
  VARIANT 10: note = X is any amino acid or variant thereof
  VARIANT 11: note = X is any amino acid or variant thereof
  VARIANT 12: note = X is any amino acid or variant thereof
  VARIANT 14: note = X is any amino acid or variant thereof
  VARIANT 15: note = X is any amino acid or variant thereof
  VARIANT 16: note = X is any amino acid or variant thereof
  VARIANT 17: note = X is any amino acid or variant thereof
  VARIANT 18: note = X is any amino acid or variant thereof
AXXXXXPLXXXXGXXXXXG

SEQ ID NO: 8 - protein, 21 aa, synthetic construct
  VARIANT 3: note = X is any amino acid or variant thereof
  VARIANT 4: note = X is any amino acid or variant thereof
  VARIANT 5: note = X is any amino acid or variant thereof
  VARIANT 14: note = X is any amino acid or variant thereof
  VARIANT 16: note = X is any amino acid or variant thereof
  VARIANT 17: note = X is any amino acid or variant thereof
  VARIANT 18: note = X is any amino acid or variant thereof
  VARIANT 19: note = X is any amino acid or variant thereof
  VARIANT 20: note = X is any amino acid or variant thereof
PIXXXYYRTWRDKXIXXXXXD

SEQ ID NO: 9 - protein, 17 aa, synthetic construct
  VARIANT 5: note = X is any amino acid or variant thereof
  VARIANT 6: note = X is any amino acid or variant thereof
  VARIANT 7: note = X is any amino acid or variant thereof
  VARIANT 8: note = X is any amino acid or variant thereof
  VARIANT 9: note = X is any amino acid or variant thereof
  VARIANT 10: note = X is any amino acid or variant thereof
  VARIANT 13: note = X is any amino acid or variant thereof
  VARIANT 16: note = X is any amino acid or variant thereof
TDIPXXXXXXSLXHVXD

SEQ ID NO: 10 - protein, 36 aa, synthetic construct
  VARIANT 2: note = X is any amino acid or variant thereof
  VARIANT 5: note = X is any amino acid or variant thereof
  VARIANT 6: note = X is any amino acid or variant thereof
  VARIANT 9: note = X is any amino acid or variant thereof
  VARIANT 12: note = X is any amino acid or variant thereof
  VARIANT 13: note = X is any amino acid or variant thereof
  VARIANT 14: note = X is any amino acid or variant thereof
  VARIANT 16: note = X is any amino acid or variant thereof
  VARIANT 18: note = X is any amino acid or variant thereof
  VARIANT 20: note = X is any amino acid or variant thereof
  VARIANT 21: note = X is any amino acid or variant thereof
  VARIANT 25: note = X is any amino acid or variant thereof
  VARIANT 27: note = X is any amino acid or variant thereof
  VARIANT 28: note = X is any amino acid or variant thereof
  VARIANT 30: note = X is any amino acid or variant thereof
  VARIANT 31: note = X is any amino acid or variant thereof
  VARIANT 32: note = X is any amino acid or variant thereof
  VARIANT 33: note = X is any amino acid or variant thereof
  VARIANT 34: note = X is any amino acid or variant thereof
  VARIANT 35: note = X is any amino acid or variant thereof
QXSDXXFWXTFXXXYXPXLXXRGTXVXXTXXXXXXL

SEQ ID NO: 11 - protein, 26 aa, synthetic construct
  VARIANT 2: note = X is any amino acid or variant thereof
  VARIANT 3: note = X is any amino acid or variant thereof
  VARIANT 4: note = X is any amino acid or variant thereof
  VARIANT 6: note = X is any amino acid or variant thereof
  VARIANT 7: note = X is any amino acid or variant thereof
  VARIANT 8: note = X is any amino acid or variant thereof
  VARIANT 9: note = X is any amino acid or variant thereof
  VARIANT 10: note = X is any amino acid or variant thereof
  VARIANT 11: note = X is any amino acid or variant thereof
  VARIANT 14: note = X is any amino acid or variant thereof
  VARIANT 15: note = X is any amino acid or variant thereof
  VARIANT 17: note = X is any amino acid or variant thereof
  VARIANT 23: note = X is any amino acid or variant thereof
YXXXAXXXXXXYVXXHXLDGLDXDME

SEQ ID NO: 12 - protein, 19 aa, synthetic construct
  VARIANT 3: note = X is any amino acid or variant thereof
  VARIANT 4: note = X is any amino acid or variant thereof
  VARIANT 6: note = X is any amino acid or variant thereof
  VARIANT 8: note = X is any amino acid or variant thereof
  VARIANT 9: note = X is any amino acid or variant thereof
  VARIANT 10: note = X is any amino acid or variant thereof
  VARIANT 12: note = X is any amino acid or variant thereof
  VARIANT 15: note = X is any amino acid or variant thereof
  VARIANT 16: note = X is any amino acid or variant thereof
  VARIANT 17: note = X is any amino acid or variant thereof
  VARIANT 18: note = X is any amino acid or variant thereof
LRXXMXAXXXLXGPXXXXN

SEQ ID NO: 13 - protein, 17 aa, synthetic construct
  VARIANT 2: note = X is any amino acid or variant thereof
  VARIANT 4: note = X is any amino acid or variant thereof
  VARIANT 6: note = X is any amino acid or variant thereof
  VARIANT 7: note = X is any amino acid or variant thereof
  VARIANT 11: note = X is any amino acid or variant thereof
  VARIANT 12: note = X is any amino acid or variant thereof
  VARIANT 13: note = X is any amino acid or variant thereof
  VARIANT 14: note = X is any amino acid or variant thereof
  VARIANT 15: note = X is any amino acid or variant thereof
  VARIANT 16: note = X is any amino acid or variant thereof
LXYXTXXNAQXXXXXXV

SEQ ID NO: 14 - protein, 12 aa, synthetic construct
  VARIANT 2: note = X is any amino acid or variant thereof
  VARIANT 3: note = X is any amino acid or variant thereof
  VARIANT 5: note = X is any amino acid or variant thereof
  VARIANT 8: note = X is any amino acid or variant thereof
  VARIANT 9: note = X is any amino acid or variant thereof
AXXVXYVXXQTY

SEQ ID NO: 15 - protein, 25 aa, synthetic construct
  VARIANT 3: note = X is any amino acid or variant thereof
  VARIANT 4: note = X is any amino acid or variant thereof
  VARIANT 6: note = X is any amino acid or variant thereof
  VARIANT 7: note = X is any amino acid or variant thereof
  VARIANT 8: note = X is any amino acid or variant thereof
  VARIANT 9: note = X is any amino acid or variant thereof
  VARIANT 14: note = X is any amino acid or variant thereof
  VARIANT 15: note = X is any amino acid or variant thereof
  VARIANT 16: note = X is any amino acid or variant thereof
  VARIANT 19: note = X is any amino acid or variant thereof
  VARIANT 23: note = X is any amino acid or variant thereof
  VARIANT 25: note = X is any amino acid or variant thereof
WNXXRXXXXSCQFXXXYAXPEEXDX

SEQ ID NO: 16 - protein, 25 aa, synthetic construct
  VARIANT 5: note = X is any amino acid or variant thereof
  VARIANT 7: note = X is any amino acid or variant thereof
  VARIANT 9: note = X is any amino acid or variant thereof
  VARIANT 11: note = X is any amino acid or variant thereof
  VARIANT 13: note = X is any amino acid or variant thereof
  VARIANT 14: note = X is any amino acid or variant thereof
  VARIANT 15: note = X is any amino acid or variant thereof
  VARIANT 16: note = X is any amino acid or variant thereof
  VARIANT 18: note = X is any amino acid or variant thereof
  VARIANT 19: note = X is any amino acid or variant thereof
  VARIANT 20: note = X is any amino acid or variant thereof
  VARIANT 22: note = X is any amino acid or variant thereof
  VARIANT 24: note = X is any amino acid or variant thereof
NRFLXAXGXGXVXXXXAXXXAXWXP

SEQ ID NO: 17 - protein, 36 aa, synthetic construct
  VARIANT 2: note = X is any amino acid or variant thereof
  VARIANT 6: note = X is any amino acid or variant thereof
  VARIANT 7: note = X is any amino acid or variant thereof
  VARIANT 8: note = X is any amino acid or variant thereof
  VARIANT 11: note = X is any amino acid or variant thereof
  VARIANT 16: note = X is any amino acid or variant thereof
  VARIANT 20: note = X is any amino acid or variant thereof
  VARIANT 21: note = X is any amino acid or variant thereof
  VARIANT 23: note = X is any amino acid or variant thereof
  VARIANT 24: note = X is any amino acid or variant thereof
  VARIANT 27: note = X is any amino acid or variant thereof
  VARIANT 28: note = X is any amino acid or variant thereof
  VARIANT 30: note = X is any amino acid or variant thereof
  VARIANT 32: note = X is any amino acid or variant thereof
  VARIANT 33: note = X is any amino acid or variant thereof
  VARIANT 34: note = X is any amino acid or variant thereof
GXKGGXXXYAXDRDGXTYDXXDXXTLXXTXFXXXKR

SEQ ID NO: 18 - protein, 14 aa, synthetic construct
  VARIANT 2: note = X is any amino acid or variant thereof
  VARIANT 3: note = X is any amino acid or variant thereof
  VARIANT 5: note = X is any amino acid or variant thereof
  VARIANT 8: note = X is any amino acid or variant thereof
  VARIANT 9: note = X is any amino acid or variant thereof
AXXVXYVXXQTYEK

SEQ ID NO: 19 - protein, 342 aa, Corynebacterium pseudotuberculosis
APAALSNAPLAASPGQADKVGAQATCAAKPIFFGYYRTWRDKAIELNDGDKWKDKLHTKLTDIPEQVDMVSLFHVPDNQKSDQRFWETFDKEYHPTLKERGTKVVRTIGAKLLLNKIKEKGLYGQSREDDSKYREIAHEVYEEYVAKHNLDGLDVDMELREVEKYTNLRWQLRKIMGAFSELMGPKAPGNAGKKPGDDGYKYLIYDTFDNAQLAQVALVADVVDYVLAQTYDKGTEESITRVWNGFRDKINSCQFLAGYAHPEENDTNRFLTAIGDVDTSGAMNVAAWKPEGGEKGGTFAYALDRDGRTYDGDDLTTLKPTDFAFTKRAIELTKGISLTDLG

SEQ ID NO: 20 - protein, 4 aa, synthetic construct
FFFD

SEQ ID NO: 21 - protein, 19 aa, synthetic construct
  VARIANT 2: note = X is any amino acid or variant thereof
  VARIANT 4: note = X is any amino acid or variant thereof
  VARIANT 13: note = X is any amino acid or variant thereof
  VARIANT 14: note = X is any amino acid or variant thereof
  VARIANT 15: note = X is any amino acid or variant thereof
  VARIANT 16: note = X is any amino acid or variant thereof
  VARIANT 17: note = X is any amino acid or variant thereof
  VARIANT 18: note = X is any amino acid or variant thereof
LXYXTFFFDNAQXXXXXXV

SEQ ID NO: 22 - protein, 4 aa, synthetic construct
PVFF

SEQ ID NO: 23 - protein, 10 aa, synthetic construct
YYRTWRDKAI

SEQ ID NO: 24 - protein, 5 aa, synthetic construct
LTDIP

SEQ ID NO: 25 - protein, 7 aa, synthetic construct
MVSLFHA

SEQ ID NO: 26 - protein, 4 aa, synthetic construct
VRTT

SEQ ID NO: 27 - protein, 11 aa, synthetic construct
HNLDGLDVDME

SEQ ID NO: 28 - protein, 4 aa, synthetic construct
GPKA

SEQ ID NO: 29 - protein, 7 aa, synthetic construct
LIYDTFD

SEQ ID NO: 30 - protein, 5 aa, synthetic construct
VDYVL

SEQ ID NO: 31 - protein, 7 aa, synthetic construct
GYAHPEE

SEQ ID NO: 32 - protein, 8 aa, synthetic construct
NRFETAIG

SEQ ID NO: 33 - protein, 7 aa, synthetic construct
DRDGRTY

SEQ ID NO: 34 - protein, 4 aa, synthetic construct
TFKR

SEQ ID NO: 35 - protein, 19 aa, synthetic construct
  VARIANT 2: note = X is any amino acid or variant thereof
  VARIANT 3: note = X is any amino acid or variant thereof
  VARIANT 4: note = X is any amino acid or variant thereof
  VARIANT 5: note = X is any amino acid or variant thereof
  VARIANT 6: note = X is any amino acid or variant thereof
  VARIANT 9: note = X is any amino acid or variant thereof
  VARIANT 10: note = X is any amino acid or variant thereof
  VARIANT 11: note = X is any amino acid or variant thereof
  VARIANT 12: note = X is any amino acid or variant thereof
  VARIANT 14: note = X is any amino acid or variant thereof
  VARIANT 15: note = X is any amino acid or variant thereof
  VARIANT 16: note = X is any amino acid or variant thereof
  VARIANT 17: note = X is any amino acid or variant thereof
  VARIANT 18: note = X is any amino acid or variant thereof
PXXXXXSLXXXXGXXXXXG

SEQ ID NO: 36 - protein, 21 aa, synthetic construct
  VARIANT 5: note = X is any amino acid or variant thereof
  VARIANT 16: note = X is any amino acid or variant thereof
  VARIANT 17: note = X is any amino acid or variant thereof
  VARIANT 18: note = X is any amino acid or variant thereof
  VARIANT 19: note = X is any amino acid or variant thereof
  VARIANT 20: note = X is any amino acid or variant thereof
PVFFXYYRTWRDKAIXXXXXD

SEQ ID NO: 37 - protein, 18 aa, synthetic construct
  VARIANT 6: note = X is any amino acid or variant thereof
  VARIANT 7: note = X is any amino acid or variant thereof
  VARIANT 9: note = X is any amino acid or variant thereof
  VARIANT 17: note = X is any amino acid or variant thereof
LTDIPXXVXMVSLFHAXN

SEQ ID NO: 38 - protein, 36 aa, synthetic construct
  VARIANT 2: note = X is any amino acid or variant thereof
  VARIANT 5: note = X is any amino acid or variant thereof
  VARIANT 6: note = X is any amino acid or variant thereof
  VARIANT 9: note = X is any amino acid or variant thereof
  VARIANT 12: note = X is any amino acid or variant thereof
  VARIANT 13: note = X is any amino acid or variant thereof
  VARIANT 14: note = X is any amino acid or variant thereof
  VARIANT 16: note = X is any amino acid or variant thereof
  VARIANT 18: note = X is any amino acid or variant thereof
  VARIANT 21: note = X is any amino acid or variant thereof
  VARIANT 25: note = X is any amino acid or variant thereof
  VARIANT 30: note = X is any amino acid or variant thereof
  VARIANT 31: note = X is any amino acid or variant thereof
  VARIANT 33: note = X is any amino acid or variant thereof
  VARIANT 34: note = X is any amino acid or variant thereof
YXSDXXFWXTFXXXYXPXLKXRGTXVRTTXXAXXLL

SEQ ID NO: 39 - protein, 29 aa, synthetic construct
  VARIANT 2: note = X is any amino acid or variant thereof
  VARIANT 3: note = X is any amino acid or variant thereof
  VARIANT 7: note = X is any amino acid or variant thereof
  VARIANT 9: note = X is any amino acid or variant thereof
  VARIANT 10: note = X is any amino acid or variant thereof
  VARIANT 11: note = X is any amino acid or variant thereof
  VARIANT 12: note = X is any amino acid or variant thereof
  VARIANT 13: note = X is any amino acid or variant thereof
  VARIANT 14: note = X is any amino acid or variant thereof
  VARIANT 17: note = X is any amino acid or variant thereof
  VARIANT 18: note = X is any amino acid or variant thereof
DXXYREXAXXXXXXYVXXHNLDGLDVDME

SEQ ID NO: 40 - protein, 21 aa, synthetic construct
  VARIANT 2: note = X is any amino acid or variant thereof
  VARIANT 6: note = X is any amino acid or variant thereof
  VARIANT 8: note = X is any amino acid or variant thereof
  VARIANT 10: note = X is any amino acid or variant thereof
  VARIANT 14: note = X is any amino acid or variant thereof
  VARIANT 19: note = X is any amino acid or variant thereof
  VARIANT 20: note = X is any amino acid or variant thereof
WXIRKXMXAXSELXGPKAXXN

SEQ ID NO: 41 - protein, 17 aa, synthetic construct
  VARIANT 4: note = X is any amino acid or variant thereof
  VARIANT 12: note = X is any amino acid or variant thereof
  VARIANT 13: note = X is any amino acid or variant thereof
  VARIANT 14: note = X is any amino acid or variant thereof
  VARIANT 15: note = X is any amino acid or variant thereof
  VARIANT 16: note = X is any amino acid or variant thereof
GYKXLIYDTFDXXXXXQ

SEQ ID NO: 42 - protein, 12 aa, synthetic construct
  VARIANT 2: note = X is any amino acid or variant thereof
  VARIANT 3: note = X is any amino acid or variant thereof
  VARIANT 9: note = X is any amino acid or variant thereof
AXXVDYVLXQTY

SEQ ID NO: 43 - protein, 25 aa, synthetic construct
  VARIANT 3: note = X is any amino acid or variant thereof
  VARIANT 4: note = X is any amino acid or variant thereof
  VARIANT 6: note = X is any amino acid or variant thereof
  VARIANT 7: note = X is any amino acid or variant thereof
  VARIANT 8: note = X is any amino acid or variant thereof
  VARIANT 9: note = X is any amino acid or variant thereof
  VARIANT 14: note = X is any amino acid or variant thereof
  VARIANT 15: note = X is any amino acid or variant thereof
  VARIANT 23: note = X is any amino acid or variant thereof
WNXXRXXXXSCQFXXGYAHPEEXDT

SEQ ID NO: 44 - protein, 25 aa, synthetic construct
  VARIANT 9: note = X is any amino acid or variant thereof
  VARIANT 11: note = X is any amino acid or variant thereof
  VARIANT 13: note = X is any amino acid or variant thereof
  VARIANT 14: note = X is any amino acid or variant thereof
  VARIANT 16: note = X is any amino acid or variant thereof
  VARIANT 19: note = X is any amino acid or variant thereof
  VARIANT 22: note = X is any amino acid or variant thereof
  VARIANT 24: note = X is any amino acid or variant thereof
NRFETAIGXGXVXXSXAMXVAXWXP

SEQ ID NO: 45 - protein, 37 aa, synthetic construct
  VARIANT 3: note = X is any amino acid or variant thereof
  VARIANT 7: note = X is any amino acid or variant thereof
  VARIANT 9: note = X is any amino acid or variant thereof
  VARIANT 12: note = X is any amino acid or variant thereof
  VARIANT 20: note = X is any amino acid or variant thereof
  VARIANT 21: note = X is any amino acid or variant thereof
  VARIANT 24: note = X is any amino acid or variant thereof
  VARIANT 25: note = X is any amino acid or variant thereof
  VARIANT 26: note = X is any amino acid or variant thereof
  VARIANT 27: note = X is any amino acid or variant thereof
  VARIANT 29: note = X is any amino acid or variant thereof
  VARIANT 33: note = X is any amino acid or variant thereof
GGXKGGXFXYAXDRDGRTYXXDDXXXXKXTDFXTFKR

SEQ ID NO: 46 - protein, 381 aa, synthetic construct
AALSNAPLAASPGQADKVGAQATCAAKPIFFGYYRTWRDKAIELNDGDKWKDKLHTKLTDIPEQVDMVSLFHVPDNQKSDQRFWETFDKEYHPTLKERGTKVVRTIGAKLLLNKIKEKGLYGQSREDDSKYREIAHEVYEEYVAKHNLDGLDVDMELREVEKYTNLRWQLRKIMGAFSELMGPKAPGNAGKKPGDDGYKYLIYDTFFFDNAQLAQVALVADVVDYVLAQTYDKGTEESITRVWNGFRDKINSCQFLAGYAHPEENDTNRFLTAIGDVDTSGAMNVAAWKPEGGEKGGTFAYALDRDGRTYDGDDLTTLKPTDFAFTKRAIELTKGISLTDLGTNGSRTPGANSSRTETSSFFNDPYYQKLMKEAGIADNTN

SEQ ID NO: 47 - protein, 414 aa, synthetic construct
MRNVPQSVSRFATVGITSAIFAASLSTVASAAPAALSNAPLAASPGQADKVGAQATCAAKPIFFGYYRTWRDKAIELNDGDKWKDKLHTKLTDIPEQVDMVSLFHVPDNQKSDQRFWETFDKEYHPTLKERGTKVVRTIGAKLLLNKIKEKGLYGQSREDDSKYREIAHEVYEEYVAKHNLDGLDVDMELREVEKYTNLRWQLRKIMGAFSELMGPKAPGNAGKKPGDDGYKYLIYDTFFKDNAQLAQVALVADVVDYVLAQTYEKGTEESITRVWNGFRDKINSCQFLAGYAHPEENDTNRFLTAIGDVDTSGAMNVAAWKPEGGEKGGTFAYALDRDGRTYDGDDLTTLKPTDFAFTKRAIELTKGISLTDLGTNGSRTPGANSSRTETSSFFNDPYYQKLMKEAGIADNTN

SEQ ID NO: 48 - protein, 4 aa, synthetic construct
KESV

SEQ ID NO: 49 - protein, 31 aa, synthetic construct
MRNVPQSVSRFATVGITSAIFAASLSTVASA

SEQ ID NO: 50 - protein, 4 aa, synthetic construct
FFKD

SEQ ID NO: 51 - protein, 4 aa, synthetic construct
TYEK

SEQ ID NO: 52 - protein, 414 aa, synthetic construct; identical to SEQ ID NO: 47

SEQ ID NO: 53 - protein, 33 aa, synthetic construct
MRNVPQSVSRFATVGITSAIFAASLSTVASAAP

SEQ ID NO: 54 - protein, 383 aa, synthetic construct
APAALSNAPLAASPGQADKVGAQATCAAKPIFFGYYRTWRDKAIELNDGDKWKDKLHTKLTDIPEQVDMVSLFHVPDNQKSDQRFWETFDKEYHPTLKERGTKVVRTIGAKLLLNKIKEKGLYGQSREDDSKYREIAHEVYEEYVAKHNLDGLDVDMELREVEKYTNLRWQLRKIMGAFSELMGPKAPGNAGKKPGDDGYKYLIYDTFFKDNAQLAQVALVADVVDYVLAQTYEKGTEESITRVWNGFRDKINSCQFLAGYAHPEENDTNRFLTAIGDVDTSGAMNVAAWKPEGGEKGGTFAYALDRDGRTYDGDDLTTLKPTDFAFTKRAIELTKGISLTDLGTNGSRTPGANSSRTETSSFFNDPYYQKLMKEAGIADNTN

SEQ ID NO: 55 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 19 aa, synthetic construct
  VARIANT 2: note = Xaa
  VARIANT 4: note = Xaa
  VARIANT 13: note = Xaa
  VARIANT 14: note = Xaa
  VARIANT 15: note = Xaa
  VARIANT 16: note = Xaa
  VARIANT 17: note = Xaa
  VARIANT 18: note = Xaa
LXYXTFFKDNAQXXXXXXV

SEQ ID NO: 57 - protein, 346 aa, Corynebacterium pseudotuberculosis
ATLSKEPLKASPGRADTVGVQTTCNAKPIFFGYYRTWRDKAIQLKDDDPWKDKLQVKLTDIPEHVNMVSLFHVEDNQKSDQQFWETFHREYQPELKKRGTRVVRTVGAQLLLNKIKDKNLYGKHVEDDYKYREIARDVYNEYVVKHNLDGLDVDMELRQVEKQLNLKWQLRKIMGAFSELMGPKAPANEGKKPDHEGYKYLIYDTFDNAQTSQVGLVADLVDYVLAQTYKKDTKESVTQVWNGFRDKINSCQFMAGYAHPEENDTNRFLTAVGEVNKSGAMQVAEWKPEGGEKGGTFAYALDRDGRTYDGDDFTTLKPTDFAFTKRAIELTTGESSTDLGKPTGSR